SEQUENCE LISTING (20 sequences)

SEQ ID NO: 1 - DNA, 40 bp, synthetic construct
  source 1..40: mol_type = other DNA
gggtctgacgctcagtggaacgaaaactcacgttaaggga

SEQ ID NO: 2 - DNA, 40 bp, synthetic construct
  source 1..40: mol_type = other DNA
ttttggtcatgagattatcaaaaaggatcttcacctagat

SEQ ID NO: 3 - DNA, 484 bp, synthetic construct
  source 1..484: mol_type = other DNA
gggtctgacgctcagtggaacgaaaactcacgttaagggagcggccgcgaagatccggcagaagaatggagtatgttgtaactaaagataacttcgtataatgtatgctatacgaagttatacagatcgtgcgatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatccctttagttacaacatactcaggtctttctcaagccgattttggtcatgagattatcaaaaaggatcttcacctagat

SEQ ID NO: 4 - DNA, 426 bp, synthetic construct
  source 1..426: mol_type = other DNA
gagctgttgacagagggtcattttcacactataatgcagtgaatcccaaacagtatgttgtacctaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcctttaggtacaacatactagaatatttgcctacagcctcctttcggaggctgttttttta

SEQ ID NO: 5 - DNA, 34 bp, synthetic construct
  source 1..34: mol_type = other DNA
accccgtagaaaagagagctgttgacagagggtc

SEQ ID NO: 6 - DNA, 39 bp, synthetic construct
  source 1..39: mol_type = other DNA
aagaagatcctttgataaaaaaacagcctccgaaaggag

SEQ ID NO: 7 - DNA, 44 bp, synthetic construct
  source 1..44: mol_type = other DNA
tgagcgtcagaccccgtagaaaagagagctgttgacagagggtc

SEQ ID NO: 8 - DNA, 49 bp, synthetic construct
  source 1..49: mol_type = other DNA
aaaggatctcaagaagatcctttgataaaaaaacagcctccgaaaggag

SEQ ID NO: 9 - DNA, 59 bp, synthetic construct
  source 1..59: mol_type = other DNA
gagttttcgttccactgagcgtcagaccccgtagaaaagagagctgttgacagagggtc

SEQ ID NO: 10 - DNA, 64 bp, synthetic construct
  source 1..64: mol_type = other DNA
attacgcgcagaaaaaaaggatctcaagaagatcctttgataaaaaaacagcctccgaaaggag

SEQ ID NO: 11 - DNA, 378 bp, synthetic construct
  source 1..378: mol_type = other DNA
atggtgcactctcagtacaatctgctctgatgccgcatagtatgttgtaactaaagatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatactttagttacaacatactgttaagccagccccgacacccgccaacacccgctgacgcg

SEQ ID NO: 12 - DNA, 553 bp, synthetic construct
  source 1..553: mol_type = other DNA
ggtcccacttgtattgtcgatcagactatcagcgtgagactacgattccatcaatgcctggaagatccggcagaagaatggagtatgttgtaactaaagataacttcgtataatgtatgctatacgaagttatacagatcgtgcgatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatccatctatgtcgggtgcggagaaagaggtaatgaaatggctttagttacaacatactcaggtctttctcaagccgatcaagggcaagtattgacatgtcgtcgtaacctgtagaacggagtaacctcggtgtgcgg

SEQ ID NO: 13 - DNA, 380 bp, synthetic construct
  source 1..380: mol_type = other DNA
gagcaaaaggccagcaaaaggccaggaaccgtaaaaaggcagtatgttgtaactaaagatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatactttagttacaacatactttttggtcatgagattatcaaaaaggatcttcacctagat

SEQ ID NO: 14 - DNA, 380 bp, synthetic construct
  source 1..380: mol_type = other DNA
gggtctgacgctcagtggaacgaaaactcacgttaagggaagtatgttgtaactaaagatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatactttagttacaacatactttttggtcatgagattatcaaaaaggatcttcacctagat

SEQ ID NO: 15 - DNA, 2300 bp, synthetic construct
  source 1..2300: mol_type = other DNA
aagtggtcctgcaactttatccgcctccatccagtctattaattgttgccgggaagctagagtaagtagttcgccagttaatagtttgcgcaacgttgttgccattgctacaggcatcgtaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctagtatgttgtaactaaagatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatactttagttacaacatactgggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcaacaaaaagcagctggctgacattttcggtgcgagtatccgtaccattcagaactggcaggaacagggaatgcccgttctgcgaggcggtggcaagggtaatgaggtgctttatgactctgccgccgtcataaaatggtatgccgaaagggatgctgaaattgagaacgaaaagctgcgccgggaggttgaagaactgcggcaggccagcgaggcagatctccagccaggaactattgagtacgaacgccatcgacttacgcgtgcgcaggccgacgcacaggaactgaagaatgccagagactccgctgaagtggtggaaaccgcattctgtactttcgtgctgtcgcggatcgcaggtgaaattgccagtattctcgacgggctccccctgtcggtgcagcggcgttttccggaactggaaaaccgacatgttgatttcctgaaacgggatatcatcaaagccatgaacaaagcagccgcgctggatgaactgataccggggttgctgagtgaatatatcgaacagtcaggttaacaggctgcggcattttgtccgcgccgggcttcgctcactgttcaggccggagccacagaccgccgttgaatgggcggatgctaattactatctcccgaaagaatccgcataccaggaagggcgctgggaaacactgccctttcacgctcgtcatcaaaatcactcgcatcaaccaaaccgttattcattcgtgattgcgcctgagcgagacgaaatacgcgatcgctgttaaaaggacaattacaaacaggaatcgaatgcaac

SEQ ID NO: 16 - DNA, 546 bp, synthetic construct
  source 1..546: mol_type = other DNA
tgggaccacggtcccacttgtattgtcgatcagactatcagcgtgagactacgattccattaaaaaaacagcctccgaaaggaggctgtaggcaaatattctagtatgttgtacctaaaggaaatgagctgacagatcgtgcgatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatccctttaggtacaacatactgtttgggattcactgcattatagtgtgaaaatgaccctctgtcaacagctcatgtcgtcgtaacctgtagaacggagttcttttccgcgggctgggatttaaaagcggcag

SEQ ID NO: 17 - DNA, 546 bp, synthetic construct
  source 1..546: mol_type = other DNA
tgggaccacggtcccacttgtattgtcgatcagactatcagcgtgagactacgattccattaaaaaaacagcctccgaaaggaggctgtaggcaaatattctagtatgttgtacctaaaggaaatgagctgacagatcgtgcgatctactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatccctttaggtacaacatactgtttgggattcactgcattatagtgtgaaaatgaccctctgtcaacagctcatgtcgtcgtaacctgtagaacggagtaacctcggtgtgcagattgtttccctccgtaaa

SEQ ID NO: 18 - DNA, 487 bp, synthetic construct
  source 1..487: mol_type = other DNA
ttttgataatctcatgaccaaaatcccttaacgtgagttttcgttccactgagcgtcagaccccgtagaaaagatgcacaccgaggttactccgttctacgggatcctgggtattaaaaagaagatctatttatttagagatctgttctattgtgatctcttattaggatcgcctaggcctgtggataacctaggtccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcttcgtccatttgacaggccatcaaaggatcttcttgagatcctttttttctgcgcgtaatctgctgcttgcaaacaaaaaaaccaccgctaccagcggt

SEQ ID NO: 19 - DNA, 100 bp, synthetic construct
  source 1..100: mol_type = other DNA
ttttgataatctcatgaccaaaatcccttaacgtgagttttcgttccactgagcgtcagaccccgtagaaaagatgcacaccgaggttactccgttctac

SEQ ID NO: 20 - DNA, 100 bp, synthetic construct
  source 1..100: mol_type = other DNA
accgctggtagcggtggtttttttgtttgcaagcagcagattacgcgcagaaaaaaaggatctcaagaagatcctttgatggcctgtcaaatggacgaag

(19) United States
(12) Patent Application Publication
(10) Pub. No.: US 2024/0352495 A1
(43) Pub. Date: Oct. 24, 2024

(54) Method for Producing Circular DNA

(71) Applicant: Moderna Enzymatics Co., Ltd., Tokyo (JP)

(72) Inventors: Masayuki Suetsugu, Tokyo (JP); Seia Nara, Tokyo (JP)

(21) Appl. No.: 18/685,259

(22) PCT Filed: Sep. 13, 2022
(86) PCT No.: PCT/JP2022/034150

§ 371 Date: Feb. 21, 2024

(30) Foreign Application Priority Data
Sep. 13, 2021 (JP) 2021-148639

(51) Int. Cl. C12P 19/34; C12N 9/12; C12N 15/11

(52) U.S. Cl. CPC C12P 19/34; C12N 9/1205; C12N 9/1217; C12N 9/1223; C12N 9/1229; C12N 15/11; C12Y 207/0104; C12Y 207/02001; C12Y 207/03002; C12Y 207/04001; C12Y 207/04006

(57) ABSTRACT

Provided is a method for producing circular DNA in which a region that is sandwiched by a region Ha and a region Hb in circular double-stranded DNA is substituted with the entirety or a portion of a linear-DNA fragment, wherein the region Hb is located downstream of the region Ha in the circular double-stranded DNA; the linear-DNA fragment is single-stranded or double-stranded linear DNA that has a homologous region that corresponds to the region Ha and a homologous region that corresponds to the region Hb, the latter homologous region being positioned downstream of the former homologous region; and the method comprising: preparing a reaction solution that contains the circular double-stranded DNA, the linear-DNA fragment, and a protein that has RecA-family-recombinase activity, and performing homologous recombination reaction by incubating the reaction solution for a predetermined period of time, thereby producing circular DNA in which the region that is from the region Ha to the region Hb in the circular double-stranded DNA is substituted with the region that is from the homologous region that corresponds to the region Ha to the homologous region that corresponds to the region Hb in the linear-DNA fragment.

### TECHNICAL FIELD

The present invention relates to a method for substituting a portion of circular DNA with a linear-DNA fragment.

The present application claims priority based on JP 2021-148639 filed in Japan on Sep. 13, 2021, and the content thereof is incorporated herein.

### BACKGROUND ART

The operation of inserting or substituting a DNA fragment into a target site of circular DNA, such as a plasmid, is the basis of genetic engineering. Generally, this operation is achieved by converting the circular DNA into linear-DNA fragments in advance and then ligating the linear-DNA fragments to each other. Linearization of the circular DNA can be achieved by, for example, cleaving a target region in the circular DNA using a restriction enzyme or the like or performing PCR amplification using the circular DNA as a template. Examples of ligation reactions between linear-DNA fragments include the Infusion method (see Patent Literature 1), the Gibson Assembly method (see Patent Literature 2 and Patent Literature 3), and the Recombination Assembly method (see Patent Literature 4).

As an example of a method for inserting linear-DNA fragments without linearizing the circular DNA, Gateway cloning using a site-specific recombination mechanism (see Non-Patent Literature 1) is well known, and reagent kits are commercially available (by Thermo Fisher). Gateway cloning requires that the circular DNA into which the linear-DNA fragments are inserted has a recombinant sequence recognized by a site-specific recombinase. In addition, the Recombineering method using a homologous recombination mechanism is known as a method for inserting or substituting a linear-DNA fragment into circular DNA in a cell (see Non-Patent Literature 2).

### CITATION LIST

### Patent Literature

Patent Literature 1: U.S. Pat. No. 7,575,860
Patent Literature 2: U.S. Pat. No. 7,776,532
Patent Literature 3: U.S. Pat. No. 8,968,999
Patent Literature 4: International Publication No. WO 2019/009361

### Non-Patent Literature

Non-Patent Literature 1: Hartley et al., Genome Research, 2000, vol. 10, pp. 1788-1795.
Non-Patent Literature 2: Copeland et al., Nature Reviews Genetics, 2001, vol. 2, pp. 769-779.
Non-Patent Literature 3: Su'etsugu et al., Nucleic Acids Research, 2017, vol. 45 (20), pp. 11525-11534.

### SUMMARY OF INVENTION

### Problem to be Solved by Invention

There has been no known method for inserting or substituting a linear-DNA fragment into a target site in circular DNA in vitro without cleaving the circular DNA or using a site-specific recombination sequence.

The main purpose of the present invention is to provide a method for producing circular DNA by directly inserting a linear-DNA fragment into the circular DNA without linearizing it—it remains circular.

### Means for Solving Problem

As a result of diligent research, the inventors of the present invention found that a linear-DNA fragment having a base sequence homologous to a target site of circular DNA at both ends can be recombined with the target site of the circular DNA in vitro by using a RecA family recombinase and, if necessary, exonuclease—this led to the completion of the present invention.

The method for producing circular DNA according to the present invention is as follows in [1] to [14].

[1] A method for producing circular DNA in which a region that is sandwiched by a region Ha and a region Hb in circular double-stranded DNA is substituted with the entirety or a portion of a linear-DNA fragment, wherein
the region Hb is located downstream of the region Ha in the circular double-stranded DNA;
the linear-DNA fragment is single-stranded or double-stranded linear DNA that has a homologous region that corresponds to the region Ha and a homologous region that corresponds to the region Hb, the latter homologous region being positioned downstream of the former homologous region; and the method comprising:
   preparing a reaction solution that contains the circular double-stranded DNA, the linear-DNA fragment, and a protein that has RecA-family-recombinase activity, and performing homologous recombination reaction by incubating the reaction solution for a predetermined period of time, thereby producing circular DNA in which the region that is from the region Ha to the region Hb in the circular double-stranded DNA is substituted with the region that is from the homologous region that corresponds to the region Ha to the homologous region that corresponds to the region Hb in the linear-DNA fragment.
      [2] The method for producing circular DNA of [1], wherein the linear-DNA fragment is double-stranded linear DNA, and at least a portion of the linear-DNA fragment is made into a single strand at the same time as or prior to the homologous recombination.
      [3] The method for producing circular DNA of [1] or [2], wherein the base-pair length of the region Ha and the base-pair length of the region Hb are 10-500 bp, respectively.
      [4] The method for producing circular DNA of any of [1] to [3], wherein the reaction solution is incubated in a temperature range of 20-48° C. in the homologous recombination.
      [5] The method for producing circular DNA of any of [1] to [4], wherein the circular double-stranded DNA prior to the homologous recombination has a nick.
      [6] The method for producing circular DNA of any of [1] to [5], wherein the linear-DNA fragment is double-stranded linear DNA, and the reaction solution further contains an exonuclease.
      [7] The method for producing circular DNA of [6], wherein the exonuclease is a 3′ to 5′ exonuclease or a 5′ to 3′ exonuclease.
      [8] The method for producing circular DNA of [6] or [7], wherein the reaction solution contains an enzyme for regenerating a nucleoside triphosphate or deoxynucleotide triphosphate and a substrate for the enzyme.
      [9] The method for producing circular DNA of [8], wherein the enzyme is creatine kinase and the substrate is creatine phosphate,
the enzyme is pyruvate kinase and the substrate is phosphoenolpyruvate,
the enzyme is acetate kinase and the substrate is acetyl phosphate,
the enzyme is a polyphosphate kinase and the substrate is a polyphosphate, or
the enzyme is a nucleoside-diphosphate kinase and the substrate is a nucleoside triphosphate.
   [10] The method for producing circular DNA of any of [1] to [9], wherein the circular DNA that is obtained by the homologous recombination is amplified.
   [11] The method for producing circular DNA of any of [1] to [10], wherein the circular DNA that is obtained by the homologous recombination contains a replication origin that can bind to an enzyme that has DnaA activity, and the method comprising:
forming a reaction mixture that contains the circular DNA that is obtained by the homologous recombination, a first enzyme group that catalyzes replication of circular DNA, a second enzyme group that catalyzes Okazaki fragment joining reaction and synthesizes two sister circular DNAs constituting a catenane, and a third enzyme group that catalyzes the separation of the two sister circular DNAs, and dNTP; and incubating the reaction mixture formed under an isothermal conditions; thereby repairing a gap and nick in the circular DNA that is obtained by the homologous recombination and amplifying the circular DNA.
   [12] The method for producing circular DNA of any of [1] to [11], wherein the linear-DNA fragment has a target gene between the homologous region that corresponds to the region Ha and the homologous region that corresponds to the region Hb, and circular DNA is produced that has the target gene inserted in the region that is sandwiched by the region Ha and the region Hb in the circular double-stranded DNA.
   [13] The method for producing circular DNA of [12], wherein the target gene is a drug resistance gene.
   [14] The method for producing circular DNA of any of [1] to [13], wherein the circular double-stranded DNA is a plasmid.

### Effect of Invention

The method for producing circular DNA according to the present invention makes it possible to obtain circular DNA that is directly recombined by the entirety or a portion of a linear-DNA fragment without linearizing circular double-stranded DNA.

### BRIEF DESCRIPTION OF DRAWINGS

FIG. 1 is a diagram schematically illustrating an embodiment of a method for preparing a double-stranded linear-DNA fragment in which a homologous region that corresponds to a region Ha and a homologous region that corresponds to a region Hb are made into a single strand.

FIG. 2 is a staining image of bands in Example 1 separated by agarose electrophoresis of a reaction solution subjected to RCR amplification following homologous recombination.

FIG. 3 is a staining image of bands in Example 2 separated by agarose electrophoresis of a reaction solution subjected to RCR amplification following homologous recombination using linear-DNA fragments having homologous regions of different lengths.

FIG. 4 is a staining image of bands in Example 3 separated by agarose electrophoresis of a reaction solution subjected to RCR amplification following homologous recombination at different reaction temperatures.

FIG. 5 is a staining image of bands in Example 4 separated by agarose electrophoresis of a reaction solution subjected to RCR amplification following homologous recombination using 5′ to 3′ exonuclease.

FIG. 6 is a staining image of bands in Example 5 separated by agarose electrophoresis of a reaction solution subjected to RCR amplification following homologous recombination using a RecA-family recombinase protein of different concentrations.

FIG. 7 is a staining image of bands in Example 6 separated by agarose electrophoresis of a reaction solution subjected to RCR amplification following homologous recombination at different reaction times.

FIG. 8 is a staining image of bands in Example 7 separated by agarose electrophoresis of a reaction solution subjected to RCR amplification following homologous recombination and a digested product obtained by digesting the amplification product in the reaction solution using a restriction enzyme EcoRI.

FIG. 9 is a staining image of bands in Example 8 separated by agarose electrophoresis of a reaction solution subjected to RCR amplification following homologous recombination.

FIG. 10 is a staining image of bands in Example 9 separated by agarose electrophoresis of a reaction solution subjected to RCR amplification following homologous recombination.

FIG. 11 is a staining image of bands in Example 10 separated by agarose electrophoresis of a reaction solution subjected to RCR amplification following homologous recombination.

### EMBODIMENTS OF INVENTION

### <Method for Producing Circular DNA>

The method for producing circular DNA according to the present invention involves producing circular DNA into which linear DNA fragments have been incorporated without linearizing circular double-stranded DNA by causing mutual homologous recombination between homologous regions of circular double-stranded DNA and a linear-DNA fragment having corresponding homologous regions in which homologous recombination mutually occurs. Specifically, a region that is from a region Ha to a region Hb in the circular double-stranded DNA is substituted with a region that is from a homologous region that corresponds to the region Ha to a homologous region that corresponds to the region Hb in the linear-DNA fragment. The linear-DNA fragment is typically single-stranded or double-stranded linear DNA having a homologous region that corresponds to the region Ha at one end or in the vicinity thereof and a homologous region that corresponds to the region Hb at the other end or in the vicinity thereof. Conventional ligation reactions between linear-DNA fragments do not target the ligation of circular DNA having no end and linear DNA, and require circular DNA to be linearized in advance. In contrast to this, in the method for producing circular DNA according to the present invention, homologous recombination is performed in the presence of a RecA-family recombinase protein, and if necessary, the linear DNA is linearized at the same time as or prior to homologous recombination, and a portion of the cyclic double-stranded DNA may be substituted with the entirety or a portion of the linear-DNA fragment as is without linearizing the circular double-stranded DNA in advance. Note that in the present specification, linearizing the circular double-stranded DNA means that both strands of the circular double-stranded DNA are cleaved to become linear DNA. Although there is a conventional technique of introducing a linear-DNA fragment randomly into a portion of circular double-stranded DNA by using a transposable element, the method for producing circular DNA according to the present invention makes it possible to substitute a desired, rather than random, site of circular double-stranded DNA with the entirety or a portion of a linear-DNA fragment.

In the present invention and the specification of the present application, the “base sequences are homologous” means that the “base sequences are identical,” and the “base sequences are complementary” means that the “base sequences are mutually complementary.” Regions where base sequences are homologous are sometimes referred to simply as “homologous regions.”

In the present invention and the specification of the present application, a “homologous region that corresponds to a region Ha” refers to a region having sufficient sequence identity to cause homologous recombination with region Ha, preferably homologous recombination using the RecA-family recombinase protein. It is preferable that the sequence identity of such a region with the region Ha is 80% or more, 85% or more, 90% or more, 95% or more, 98% or more, 99% or more, or 100%. That is, the “homologous region that corresponds to the region Ha” includes the “homologous region of the region Ha.” The same applies for the “homologous region that corresponds to the region Hb.”

Specifically, the method for producing circular DNA according to the present invention involves preparing a reaction solution that contains the circular double-stranded DNA, the linear-DNA fragment, and a protein that has RecA-family-recombinase activity (hereinafter, this may be referred to as “RecA-family recombinase protein”), and performing homologous recombination by incubating the reaction solution for a predetermined period of time, the homologous recombination producing circular DNA in which the region that is from the region Ha to the region Hb in the circular double-stranded DNA is substituted with the region that is from the homologous region that corresponds to the region Ha to the homologous region that corresponds to the region Hb in the linear-DNA fragment. Homologous recombination occurs for any strand of circular double-stranded DNA in which a corresponding homologous region is present. In the present invention, using the RecA-family recombinase protein makes it possible to effect homologous recombination with a linear-DNA fragment without linearizing the circular double-stranded DNA—it remains circular. Note that thereafter, the circular double-stranded DNA prior to being substituted by the entirety or a portion of the linear-DNA fragment may be referred to as “pre-substituted circular DNA,” and the circular DNA substituted by the entirety or a portion of the linear DNA fragment may be referred to as “recombinant circular DNA.”

In the present invention, recombinant circular DNA having a linear-DNA fragment inserted into the pre-substituted circular DNA is produced by homologous recombination occurring in the homologous region that corresponds to the region Ha in the pre-substituted circular DNA and the region Ha in the linear-DNA fragment and in the homologous region that corresponds to the region Hb in the pre-substituted circular DNA and the region Hb in the linear-DNA fragment. In the recombinant circular DNA, a region that is from the region Ha to the region Hb in the pre-substituted circular DNA is substituted with a region that is from the homologous region that corresponds to the region Ha to the homologous region that corresponds to the region Hb in the linear-DNA fragment.

In the pre-substituted circular DNA, the region Hb is located downstream of the region Ha, and in the linear-DNA fragment, the region Hb is located downstream of the region Ha. Herein, “the region Hb is located downstream of the region Ha” means that the region Hb is present on the 3′ end side of the region Ha in the base sequence of one strand of the circular double-stranded DNA. As long as the linear-DNA fragment has a homologous region that corresponds to the region Hb downstream of the homologous region that corresponds to the region Ha, the region Hb may be present on the 3′ end side of the region Ha in the base sequence of any of the strands of the double-stranded DNA.

When the region Hb is adjacent to downstream of the region Ha in the pre-substituted circular DNA, recombinant circular DNA is obtained in which a region between the homologous region that corresponds to the region Ha and the homologous region that corresponds to the region Hb of the linear-DNA fragment is inserted between the region Ha and the region Hb of the pre-substituted circular DNA. On the other hand, when the region Ha and the region Hb are not adjacent to each other in the pre-substituted circular DNA, recombinant circular DNA is obtained in which the region between the region Ha and the region Hb of the pre-substituted circular DNA is substituted by the region between the homologous region that corresponds to the region Ha and the homologous region that corresponds to the region Hb of the linear-DNA fragment.

Subsequently, the region from the upstream end of the homologous region that corresponds to the region Ha to the downstream end of the homologous region that corresponds to the region Hb in the linear-DNA fragment is sometimes referred to as a “target region.” Furthermore, the region from the upstream end of the region Ha to the downstream end of the region Hb in the pre-substituted circular DNA is sometimes referred to as a “substitution region.”

The base sequences of the region Ha and the region Hb used in homologous recombination may be any base sequence as long as it allows the specific hybridization of single strands in the reaction solution, and the base pair (bp) length, GC ratio, and the like may generally be determined as appropriate by referring to methods for designing probes and primers. Generally, in homologous recombination, in order to suppress nonspecific hybridization and effect desired homologous recombination, the base pair lengths of the region Ha and the region Hb (and the corresponding homologous regions) need to have a certain length, but if the base pair lengths of these regions are too long, there is a risk that the reaction efficiency will decrease.

In the present invention, the base pair lengths of the region Ha and the region Hb are preferably 10 base pairs (bp) or more, more preferably 15 bp or more, and more preferably 20 bp or more. Furthermore, the base pair lengths of the region Ha and the region Hb are preferably 500 bp or less, more preferably 300 bp or less, even more preferably 200 bp or less, and further preferably 150 bp or less. Note that the lengths and base sequences of the region Ha and the region Hb may be the same or may be different from each other.

In the present invention, the base pair lengths of the homologous region that corresponds to the region Ha and the homologous region that corresponds to the region Hb are not limited so long as homologous recombination occurs with each of the region Ha and the region Hb; however, 10 base pairs (bp) or more are preferable, 15 bp or more are more preferable, and 20 bp or more are even more preferable. Furthermore, the base pair lengths of the homologous region that corresponds to the region Ha and the homologous region that corresponds to the region Hb are preferably 500 bp or less, more preferably 300 bp or less, even more preferably 200 bp or less, and further preferably 150 bp or less. Note that the lengths and base sequences of the homologous region that corresponds to the region Ha and the homologous region that corresponds to the region Hb may be the same or may be different from each other.

The length and base sequence of the target region in the linear-DNA fragment used in the present invention are not particularly limited. For example, the length of the target region in the linear-DNA fragment may be the same as the length of the substitution region in the pre-substituted circular DNA, or may be shorter or longer. For example, the length of the target region in the linear-DNA fragment may be 100 base lengths or longer, and may be preferably 200 base lengths or longer. The length of the target region in the linear-DNA fragment may be preferably 300,000 base lengths or longer, more preferably 500,000 base lengths or longer, even more preferably 1,000,000 base lengths or longer, and further preferably 2,000,000 base lengths or longer.

Furthermore, the length of the target region in the linear-DNA fragment may be of a length such that the length of the recombinant circular DNA is 1.5 times the length of the pre-substituted circular DNA or longer—for example, about two times longer. When a linear-DNA fragment shorter than the length of the substitution region in the pre-substituted circular DNA is used, the length of the recombinant circular DNA is shorter than the length of the pre-substituted circular DNA, and the recombinant circular DNA having a length that is 75% or less than the length of the pre-substituted circular DNA—for example, around 50%—may be produced.

In the linear-DNA fragment, the homologous region that corresponds to the region Hb may be present downstream of the homologous region that corresponds to the region Ha, may be present at the end of the linear DNA fragment or in the vicinity thereof, or may be present elsewhere. In the present invention, it is preferable that the homologous region that corresponds to the region Ha is positioned at the end on the upstream side of the linear-DNA fragment or in the vicinity thereof, and it is preferable that the homologous region that corresponds to the region Hb is positioned at the end on the downstream side of the linear-DNA fragment or in the vicinity thereof. For example, the end base on the upstream side of the homologous region that corresponds to the region Ha is preferably within 300 bases from the end on the upstream side of the linear-DNA fragment, more preferably within 100 bases, even more preferably within 30 bases, and further preferably within 10 bases. The end base on the downstream side of the homologous region that corresponds to the region Hb is preferably within 300 bases from the end on the downstream side of the linear-DNA fragment, more preferably within 100 bases, more even preferably within 30 bases, and further preferably within 10 bases.

The linear-DNA fragment used in the present invention may be a single-stranded linear-DNA fragment or may be a double-stranded linear-DNA fragment. Furthermore, in the case of a double-stranded linear-DNA fragment, both ends may be blunt ends, both ends may be sticky ends, or one end may be a blunt end and the other end may be a sticky end.

In the linear-DNA fragment, when the homologous region that corresponds to the region Ha is in a single-stranded state, the region in the single-stranded state acts on the region Ha of the pre-substituted circular DNA in a double-stranded state in the presence of the RecA-family recombinase protein to cause homologous recombination in the region Ha of the pre-substituted circular DNA and the linear-DNA fragment. Similarly, in the linear-DNA fragment, when the homologous region that corresponds to the region Hb is in a single-stranded state, the region in the single-stranded acts on the region Hb of the pre-substituted circular DNA in a double-stranded state in the presence of the RecA-family recombinase protein to cause homologous recombination in the region Hb of the pre-substituted circular DNA and the linear-DNA fragment, and recombinant circular DNA is obtained.

When the linear-DNA fragment is a single-stranded linear-DNA fragment, the single-stranded linear-DNA fragment has homologous regions that respectively correspond to region Ha and the region Hb of one strand of the circular double-stranded DNA, allowing homologous recombination to be carried out.

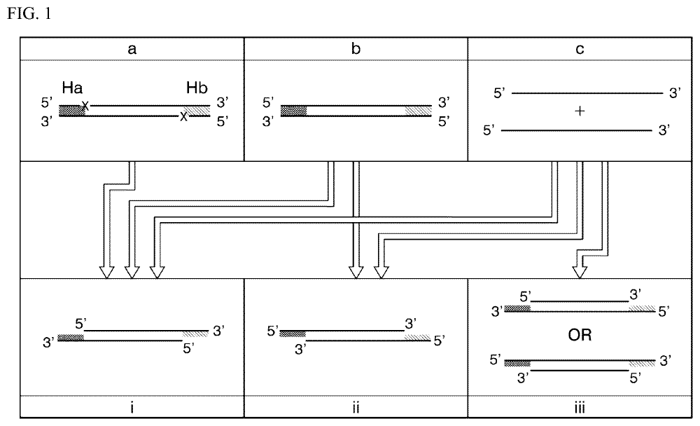

When the linear-DNA fragment is a double-stranded linear-DNA fragment, the homologous region that corresponds to the region Ha and the homologous region that corresponds to the region Hb in the double-stranded linear DNA fragment are both required to be in a single-stranded state during homologous recombination. Examples of methods for preparing such a DNA fragment include those illustrated in FIG. 1. Each will be described below.

When the linear-DNA fragment is a linear double-stranded DNA fragment and the homologous region that corresponds to the region Ha and/or the homologous region that corresponds to the region Hb is double-stranded DNA (FIG. 1, (b)), both the homologous region that corresponds to the region Ha and the homologous region that corresponds to the region Hb in the linear DNA fragment and an exonuclease are used to create a single-stranded state at the same time as or prior to the homologous recombination. A 5′ to 3′ exonuclease may expose a single strand at the 3′ end (in FIG. 1, (b) to (i)), and a 3′ to 5′ exonuclease may expose a single strand at the 5′ end (in FIG. 1, (b) to (ii)). In this case, for example, the exonuclease may also be contained in the reaction solution. From the perspective of efficiency of exonuclease treatment, when both the homologous region that corresponds to the region Ha and the homologous region that corresponds to the region Hb in the linear-DNA fragment are in a double-stranded state, it is preferable that the homologous region that corresponds to the region Ha and the homologous region that corresponds to the region Hb is on the upstream end of the linear double-stranded DNA fragment and on the downstream end of the linear double-stranded DNA fragment, respectively.

A linear double-stranded DNA fragment in which the homologous region that corresponds to the region Ha and the homologous region that corresponds to the region Hb are in a single-stranded state may also be prepared by a method where linear double-stranded DNA is prepared by PCR, or the like using a primer containing a non-standard base (X), such as dUTP (FIG. 1, (a)), after which gaps or nicks are created in the non-standard base and thermal dissociation of the primer portion exposes a single strand at the 3′ end (FIG. 1, (a)=> (i)). Homologous recombination may also be carried out by adding to the reaction solution for homologous recombination in a single-stranded state in the same manner as when exonuclease is added to the reaction solution. The non-standard base (X) and the creation of gaps or nicks therein may be performed by a known method, such as using a defined enzyme—for example, these may be performed by a method using thermal denaturation treatment at a temperature higher than the annealing temperature, or USER (registered trademark) (Uracil-Specific Excision Reagent). New England BioLabs: [online] https://international.neb.com/applications/cloning-and-synthetic-biology/user-cloning) enzyme. When the USER (registered trademark) enzyme is used, the homologous region that corresponds to the region Ha and the homologous region that corresponds to the region Hb in the linear-DNA fragment have uracil as a base; first, uracil-DNA glycosylase removes the uracil base and then the action of endonuclease VIII forms gaps in the uracil base portion. Thereafter, thermal dissociation of the region between the gaps causes the single-stranded formation of a defined region of the linear-DNA fragment. In consideration of the length of the region to be made into a single strand, a non-standard base may be incorporated into one or more sites.

Furthermore, hybridizing single-stranded DNAs with partially complementary sequences makes it possible to obtain double-stranded linear-DNA fragments in which both ends are sticky ends or one end is a sticky end (FIG. 1, (c)). For example, depending on the site and the length of the complementary sequence, a fragment where a single strand at the 3′ end is exposed (FIG. 1, (c)=> (i)), a fragment where a single strand at the 5′ end is exposed (FIG. 1, (c)=> (ii)), or a fragment where a single strand is exposed only at one end portion of a double strand (FIG. 1, (c)=> (iii)) may be obtained.

In the linear-DNA fragment, when the homologous region that corresponds to the region Ha is in a single-stranded state, the region in the single-stranded state acts on the region Ha of the pre-substituted circular DNA in a double-stranded state in the presence of the RecA-family recombinase protein to cause homologous recombination in the region Ha and the region Hb of the pre-substituted circular DNA and the linear-DNA fragment, and recombinant circular DNA is obtained. Similarly, in the linear-DNA fragment, when the homologous region that corresponds to the region Hb is in a single-stranded state, the region in the single-stranded state acts on the region Hb of the double-stranded pre-substituted circular DNA in the presence of the RecA-family recombinase protein to cause homologous recombination in the region Ha and the region Hb of the pre-substituted circular DNA and the linear-DNA fragment, and recombinant circular DNA is obtained.

When the linear DNA fragment is a double-stranded linear DNA fragment where both ends are sticky ends and both of the two sticky ends are present in one strand constituting the double-stranded linear DNA fragment, homologous recombination may be carried out by causing one sticky end of the double-stranded linear-DNA fragment to have a homologous region that corresponds to the region Ha and causing the other sticky end to have a homologous region that corresponds to the region Hb.

Even when the linear DNA fragment is a double-stranded linear DNA fragment where both ends are sticky ends and the two sticky ends are present in another strand constituting the double-stranded linear DNA fragment, homologous recombination may be carried out by causing one sticky end of the double-stranded linear-DNA fragment to have a homologous region that corresponds to the region Ha and causing the other sticky end to have a homologous region that corresponds to the region Hb.

In the method for producing circular DNA according to the present invention, the linear-DNA fragment subjected to homologous recombination with the pre-substituted circular DNA may be a single fragment, or two or more linear-DNA fragments may be subjected to homologous recombination with the pre-substituted circular DNA in a single reaction. For example, circular double-stranded DNA and a plurality of types of linear-DNA fragments having different regions between the homologous region that corresponds to the region Ha and the homologous region that corresponds to the region Hb may be used. Furthermore, a plurality of types of combinations of circular double-stranded DNAs and linear-DNA fragments may be used. That is, a plurality of types of circular double-stranded DNAs having different regions Ha and Hb and the plurality of types of linear-DNA fragments that correspond thereto may be used. Moreover, a plurality of types of combinations of circular double-stranded DNAs and linear-DNA fragments may be used having different regions Ha and Hb, and in addition, a plurality of types of linear-DNA fragments having different regions between the homologous region that corresponds to the region Ha and the homologous region that corresponds to the region Hb may be used. Thus, a plurality of types of circular DNAs may be produced at once.

In the method for producing circular DNA according to the present invention, in the pre-substituted circular DNA, the regions Ha and Hb may have a double-stranded structure formed by the hybridization of two single-stranded DNAs. That is, the pre-substituted circular DNA may be a complete circular double-stranded DNA without gaps or nicks, or may be a circular double-stranded DNA in which one or a plurality of locations have a single-stranded structure. In one aspect of the present invention, the circular double-stranded DNA prior to homologous recombination has nicks.

The molar ratio of the pre-substituted circular DNA to the linear-DNA fragment contained in the reaction solution is preferably equal to the ratio of the number of molecules of each DNA fragment constituting the desired recombinant circular DNA. The homologous recombination may be more efficiently performed by aligning the number of molecules of the DNA fragments in the reaction system at the start of the reaction. For example, it is preferable that the molar concentrations of the pre-substituted circular DNA and the linear-DNA fragment contained in the reaction solution are equal to each other.

The amounts of the pre-substituted circular DNA and linear-DNA fragments included in the reaction solution are not particularly limited. Because a sufficient amount of recombinant circular DNA is easily obtained, the concentration of the pre-substituted circular DNA and the linear-DNA fragments contained in the reaction solution at the start of the reaction is preferably 0.4 pM or more, more preferably 4 pM or more, and even more preferably 40 pM or more. For higher homologous recombination efficiency, the total concentration of the pre-substituted circular DNA and the linear-DNA fragments contained in the reaction solution at the start of the reaction is preferably 100 nM or less, more preferably 40 nM or less, even more preferably 4 nM or less, and particularly preferably 0.4 nM or less.

In the method for producing circular DNA according to the present invention, the size of the recombinant circular DNA obtained by the reaction is not particularly limited. Since the large-sized recombinant circular DNA may be obtained without linearization, the size of the obtained recombinant circular DNA is preferably, for example, 500 base lengths or longer, more preferably 1,000 base lengths or longer, even more preferably 2,000 base lengths or longer, and further preferably 4,000 base lengths or longer. The method for producing circular DNA according to the present invention makes it possible to obtain recombinant circular DNA having a base length of 300,000 or longer, preferably 500,000 or longer, more preferably 1,000,000 or longer, and even more preferably 2,000,000 or longer.

The exonuclease used in the present invention is an enzyme that sequentially hydrolyzes the linear DNA from the 3′ end or the 5′ end. The type or biological origin of the exonuclease used in the present invention are not particularly limited as long as it has an enzymatic activity that sequentially hydrolyzes linear DNA from the 3′ end or the 5′ end. Examples of the enzyme that sequentially hydrolyzes from the 3′ end (3′ to 5′ exonuclease) include a linear double-stranded DNA-specific 3′ to 5′ exonuclease such as an exonuclease III family type AP (apurinic/apyrimidinic) endonuclease or the like, and a single-stranded DNA-specific 3′ to 5′ exonuclease such as a DnaQ superfamily protein or the like. Examples of the exonuclease III family type AP endonuclease include exonuclease III (derived from Escherichia coli), ExoA (Bacillus subtilis homolog of exonuclease III), Mth212 (Archaeal homolog of exonuclease III), and AP endonuclease I (human homolog of exonuclease III). Examples of the DnaQ superfamily protein include exonuclease I (derived from Escherichia coli), exonuclease T (Exo T) (also known as RNase T), exonuclease X, DNA polymerase III epsilon subunit (DNA polymerase III epsilon subunit), DNA polymerase I, DNA polymerase II, T7 DNA polymerase, T4 DNA polymerase, Klenow DNA polymerase 5, Phi29 DNA polymerase, ribonuclease III (RNase D), and oligoribonuclease (ORN). Lambda exonuclease, exonuclease VIII, T5 exonuclease, T7 exonuclease, RecJ exonuclease, or the like may be used as the enzyme that successively hydrolyzes from the 5′ end (5′ to 3′ exonuclease).

In one aspect, for the exonuclease used in the present invention, an exonuclease III family type AP exonuclease such as exonuclease III or the like is preferable as the 3′ to 5′ exonuclease, and T5 exonuclease is preferable as the 5′ to 3′ exonuclease from the perspective of the favorable balance between the processibility of shearing of the linear double-stranded DNA fragment and the reaction efficiency in the presence of the RecA-family recombinase protein.

For the concentration of the exonuclease in the reaction solution in the present invention, at the start of the reaction, for example, 1 to 1,000 mU/μL is preferable, 5 to 1,000 mU/μL is more preferable, 5 to 500 mU/μL is even more preferable, and 10 to 150 mU/μL is further preferable. In particular, in the case where the exonuclease is a linear double-stranded DNA-specific 3′ to 5′ exonuclease, the concentration of the linear double-stranded DNA-specific 3′ to 5′ exonuclease in the reaction solution at the start of the reaction is, for example, preferably 5 to 500 mU/μL, more preferably 5 to 250 mU/μL, even more preferably 5 to 150 mU/μL, and further preferably 10 to 150 mU/μL. Furthermore, in the case where the exonuclease is a single-stranded DNA-specific 3′ to 5′ exonuclease, the concentration of the linear double-stranded DNA-specific 3′ to 5′ exonuclease in the reaction solution at the start of the reaction is preferably 1 to 10,000 mU/μL, more preferably 100 to 5,000 mU/μL, and even more preferably 200 to 2,000 mU/μL.

In the present invention and the specification of the present application, RecA-family recombinase protein means a protein that forms a filament by polymerizing on DNA in a single-stranded or double-stranded state, has hydrolytic activity against nucleoside triphosphates such as ATP (adenosine triphosphate) or the like, and has a function (RecA-family-recombinase activity) of searching homologous regions to perform homologous recombination. Examples of the RecA-family recombinase protein include a prokaryotic RecA homolog, a bacteriophage RecA homolog, an Archaeal RecA homolog, a eukaryotic RecA homolog, and the like. Examples of the prokaryotic RecA homolog include Escherichia coli RecA; a RecA derived from an extreme thermophile such as Thermus bacteria, including Thermus thermophiles and Thermus aquaticus, Thermococcus bacteria, Pyrococcus bacteria, Thermotoga bacteria, or the like; a RecA derived from radiation-resistant bacteria such as Deinococcus radiodurans; or the like. Examples of the bacteriophage RecA homolog include T4 phage UvsX or the like, examples of the Archaeal RecA homolog include RadA or the like, and examples of the eukaryotic RecA homolog include Rad51 and its paralogs, Dcm1, or the like. The amino acid sequence of these RecA homologs may be obtained from a database such as NCBI (http://www.ncbi.nlm.nih.gov/).

The RecA-family recombinase protein used in the present invention may be a wild-type protein, or may be a variant that carries RecA-family-recombinase activity wherein a mutation in which 1 to 30, preferably 1 to 10, and more preferably 1 to 5 amino acids are deleted, added, or substituted is introduced into a wild-type protein. Examples of the variant include a variant in which an amino acid substitution mutation that accelerates the function of searching for a homologous region in a wild-type protein is introduced, a variant in which various tags are added to the N-end or C-end of a wild-type protein, a variant in which heat resistance is improved (International Publication No. WO 2016/013592), or the like. Examples of the tags that may be used include those widely used in the expression or purification of a recombinant protein, such as His tag, HA (hemagglutinin) tag, Myc tag, and Flag tag. This may be a variant carrying RecA-family-recombinase activity, wherein a mutation in which one or more—for example, 1 to 30, preferably 1 to 10, and more preferably 1 to 5—amino acids are deleted, added, or substituted is introduced into the known variant. The wild-type RecA-family recombinase protein is a protein composed of the same amino acid sequence as that of the RecA-family recombinase protein carried in an organism isolated from nature.

A variant that carries the RecA-family recombinase protein is preferable as the RecA-family-recombinase activity used in the present invention. Examples of the variant include a F203W mutant in which phenylalanine, an amino acid residue at position 203 in Escherichia coli RecA, is substituted with tryptophan, and a variant in which the phenylalanine that corresponds to phenylalanine at position 203 in Escherichia coli RecA, among various RecA homologs, is substituted with tryptophan.

In the present invention, the amount of RecA-family recombinase protein in the reaction solution is not particularly limited. For the concentration of the RecA-family recombinase protein in the reaction solution in the present invention, at the start of the reaction, for example, 0.01 to 100 μM is preferable, 0.1 to 100 μM is more preferable, 0.1 to 50 μM is even more preferable, 0.5 to 10 μM is further preferable, and 1.0 to 5.0 μM is particularly preferable.

A nucleoside triphosphate or a deoxynucleotide triphosphate is required for the RecA-family recombinase protein to exhibit RecA-family-recombinase activity. Therefore, the reaction solution in the present invention contains at least one of a nucleoside triphosphate and a deoxynucleotide triphosphate. It is preferable to use one or more types selected from a group composed of ATP, GTP (guanosine triphosphate), CTP (cytidine triphosphate), UTP (uridine triphosphate), and m5UTP (5-methyluridine triphosphate) as the nucleoside triphosphate contained in the reaction solution in the present invention, and it is particularly preferable to use ATP. It is preferable to use one or more types selected from a group composed of dATP (deoxyadenosine triphosphate), dGTP (deoxyguanosine triphosphate), dCTP (deoxycytidine triphosphate), and dTTP (deoxythymidine triphosphate) as the deoxynucleotide triphosphate contained in the reaction solution in the present invention, and it is particularly preferable to use dATP. The total amount of the nucleoside triphosphate and the deoxynucleotide triphosphate contained in the reaction solution is not particularly limited as long as the amount is sufficient for the RecA-family recombinase protein to exhibit RecA-family-recombinase activity. The concentration of the nucleoside triphosphate or the concentration of the deoxynucleotide triphosphate in the reaction solution in the present invention is preferably, for example, 1 μM or more, more preferably 10 UM or more, and further preferably 30 μM or more at the start of the reaction. On the other hand, when the concentration of the nucleoside triphosphate in the reaction solution is too high, homologous recombination efficiency may, on the contrary, decrease. Therefore, the concentration of the nucleoside triphosphate or the concentration of the deoxynucleotide triphosphate in the reaction solution at the start of homologous recombination is preferably 1,000 μM or less, more preferably 500 μM or less, and even more preferably 300 μM or less.

Magnesium ions (Mg²⁺) are necessary for the RecA-family recombinase protein to exhibit RecA-family-recombinase activity and for the exonuclease to exhibit exonuclease activity. Therefore, the reaction solution in the present invention contains a magnesium ion source. The magnesium ion source is a substance which imparts magnesium ions into the reaction solution. Examples thereof include magnesium salts such as magnesium acetate [Mg(OAc)₂], magnesium chloride [MgCl₂], magnesium sulfate [MgSO₄], and the like. The preferred magnesium ion source is magnesium acetate.

The concentration of the magnesium ion source in the reaction solution in the present invention is not particularly limited, as long as it is of a concentration at which the RecA-family recombinase protein is capable of exhibiting RecA-family-recombinase activity and the exonuclease is capable of exhibiting exonuclease activity. The concentration of the magnesium ion source in the reaction solution at the start of the reaction is preferably, for example, 0.5 mM or more and more preferably 1 mM or more. On the other hand, when the concentration of the magnesium ion source in the reaction solution is too high, exonuclease activity becomes too strong, and homologous recombination efficiency may, on the contrary, decrease. Therefore, the concentration of the magnesium ion source concentration in the reaction solution at the start of the reaction is, for example, preferably 20 mM or less, more preferably 15 mM or less, even more preferably 12 mM or less, and further preferably 10 mM or less.

The reaction solution for homologous recombination in the present invention is prepared by, for example, adding the pre-substituted circular DNA, the linear-DNA fragment, and the RecA-family recombinase protein to a buffer solution, and if necessary, further adding the exonuclease, at least one of the nucleoside triphosphate and the deoxynucleotide triphosphate, and the magnesium ion source. The buffer solution is not particularly limited as long as it is a buffer solution suitable for use at pH 7 to 9, preferably pH 8. Examples include Tris-HCl, Tris-OAc, Hepes-KOH, phosphate buffer, MOPS-NaOH, Tricine-HCl, and the like. The preferred buffer solution is Tris-HCl or Tris-OAc. The concentration of the buffer solution may be selected as appropriate by a person having ordinary skill in the art, and while there is no particular limitation, for example, a concentration of 10 mM to 100 mM, preferably 10 mM to 50 mM, and more preferably 20 mM may be selected in the case of Tris-HCl or Tris-OAc.

When UvsX is used as the RecA-family recombinase protein in the present invention, it is preferable that the reaction solution for homologous recombination further contains T4 phage UvsY. UvsY is a mediator of homologous recombination in T4 phage. In T4 phage, first, single-stranded DNA binds to gp32 (a single-stranded DNA binding protein) to form a single-stranded DNA-gp32 complex. Next, by replacing gp32 in the complex with uvsX, the single-stranded DNA binds to uvsX to perform homologous recombination. UvsY destabilizes the interaction of single-stranded DNA-gp32 and stabilizes the interaction of single-stranded DNA-uvsX, thereby promoting the binding of single-stranded DNA to uvsX and in turn promoting homologous recombination (Bleuit et al., Proceedings of the National Academy of Sciences of the United States of America, 2001, vol. 98 (15), pp. 8298-8305). Homologous recombination efficiency is further promoted in the present invention by using UvsY in combination with UvsX.

When the exonuclease is used in the present invention, the reaction solution in which the homologous recombination is carried out preferably further contains an enzyme for regenerating the nucleoside triphosphate or the deoxynucleotide triphosphate and a substrate thereof. Being able to regenerate the nucleoside triphosphate or the deoxynucleotide triphosphate in the reaction solution makes it possible to carry out homologous recombination more efficiently. Examples of combinations of the enzyme for regenerating the nucleoside triphosphate or the deoxynucleotide triphosphate and the substrate thereof include a combination of creatine kinase and creatine phosphate, a combination of pyruvate kinase and phosphoenolpyruvate, a combination of acetate kinase and acetyl phosphate, a combination of polyphosphate kinase and polyphosphate, and a combination of nucleoside-diphosphate kinase and nucleoside triphosphate. The nucleoside triphosphate constituting a substrate of the nucleoside-diphosphate kinase (phosphate supply source) may be any of ATP, GTP, CTP, and UTP. Myokinase is another example of the regenerating enzyme.

The concentration of the nucleoside triphosphate regenerating enzyme and the substrate thereof in the reaction solution in which homologous recombination is carried out in the present invention is not particularly limited as long as the concentration is sufficient for enabling the nucleoside triphosphate to be regenerated during homologous recombination in the reaction solution. For example, when creatine kinase and creatine phosphate are used, the concentration of creatine kinase to be contained in the reaction solution in which homologous recombination is carried out in the present invention is preferably 1 to 1,000 ng/μL, more preferably 5 to 1,000 ng/μL, even more preferably 5 to 500 ng/μL, and further preferably 5 to 250 ng/μL, and the concentration of creatine phosphate is preferably 0.4 to 20 mM, more preferably 0.4 to 10 mM, and even more preferably 1 to 7 mM.

A substance capable of inhibiting the formation of a secondary structure of single-stranded DNA and promoting specific hybridization may be added to the reaction solution in which homologous recombination is carried out in the present invention. Examples of the substance include dimethyl sulfoxide (DMSO) and tetramethylammonium chloride (TMAC). DMSO has an effect of inhibiting the formation of a secondary structure of a base pair rich in GC. TMAC has an effect of promoting specific hybridization. When the reaction solution in which homologous recombination is carried out in the present invention contains a substance capable of inhibiting the formation of a secondary structure of single-stranded DNA and promoting specific hybridization, the concentration of the substance is not particularly limited as long as the concentration enables the substance to exert a homologous recombination-promoting effect. For example, when DMSO is used as the substance, the concentration of DMSO contained in the reaction solution in which homologous recombination is carried out in the present invention is preferably 5 to 30% by volume, more preferably 8 to 25% by volume, and even more preferably 8 to 20% by volume. When TMAC is used as the substance, the concentration of TMAC contained in the reaction solution in which homologous recombination is carried out in the present invention is preferably 60 to 300 mM, more preferably 100 to 250 mM, and even more preferably 100 to 200 mM.

A substance having a polymer clouding effect may also be added to the reaction solution in which homologous recombination is carried out in the present invention. The polymer clouding effect may enhance the interaction between DNA molecules and promote homologous recombination of DNA fragments. Examples of the substance include polyethylene glycol (PEG) 200 to 20000, polyvinyl alcohol (PVA) 200 to 20000, dextran 40 to 70, ficoll 70, and bovine serum albumin (BSA). When the reaction solution in which homologous recombination is carried out in the present invention contains a substance having the polymer clouding effect, the concentration of the substance is not particularly limited as long as the concentration enables the substance to exert a homologous recombination-promoting effect. For example, when PEG 8000 is used as the substance, the concentration of PEG 8000 contained in the reaction solution in which homologous recombination is carried out in the present invention is preferably 2 to 20% by volume, more preferably 2 to 10% by volume, and even more preferably 4 to 6% by volume.

An alkali metal ion source may also be contained in the reaction solution in which homologous recombination is carried out in the present invention. The alkali metal ion source is a substance which imparts alkali metals into the reaction solution. Sodium ions (Na) or potassium ions (K) are preferable as the alkali metal ions contained in the reaction solution in which homologous recombination is carried out in the present invention. Examples of the alkali metal ion source include potassium glutamate [KGlu], potassium aspartate, potassium chloride, potassium acetate [KOAc], sodium glutamate, sodium aspartate, sodium chloride, and sodium acetate. Potassium glutamate or potassium acetate is preferable as the alkali metal ion source contained in the reaction solution in which homologous recombination is carried out in the present invention, and potassium glutamate is particularly preferable because it improves the efficiency of homologous recombination. The concentration of the alkali metal ion source in the reaction solution at the start of the reaction is not particularly limited, and, for example, the concentration may be adjusted to where the alkali metal ions are imparted into the reaction solution at preferably 10 mM or more, more preferably in a range of 30 to 300 mM, and even more preferably in a range of 50 to 150 mM.

A reducing agent may also be contained in the reaction solution in which homologous recombination is carried out in the present invention. Examples of the reducing agent include dithiothreitol (DTT), β-mercaptoethanol (2-mercaptoethanol), tris(2-carboxyethyl) phosphine (TCEP), and glutathione. The preferable reducing agent is DTT. The reducing agent may be contained in the reaction solution in an amount of 1.0 to 15.0 mM, and preferably 2.0 to 10.0 mM.

In the method for producing DNA according to the present invention, homologous recombination is carried out by incubating for a predetermined period of time a reaction solution prepared by adding to a buffer solution the pre-substituted circular DNA, the linear-DNA fragment, the RecA-family recombinase protein, the nucleoside triphosphate, the magnesium ion source, and, if necessary, one or more selected from the group composed of the exonuclease, a set of nucleoside triphosphate regenerating enzymes and substrate thereof, the substance capable of inhibiting the formation of a secondary structure of single-stranded DNA and promoting specific hybridization, the substance having a polymer clouding effect, the alkali metal ion source, and the reducing agent under isothermal conditions at a temperature at which the RecA-family recombinase protein and the exonuclease in the reaction solution are capable of exhibiting their respective enzyme activities. The reaction temperature of the homologous recombination is preferably in a temperature range of 20 to 48° C., and more preferably in a temperature range of 24 to 42° C. In particular, when the length of the region Ha or the region Hb is 50 bases or more, the reaction temperature of homologous recombination is preferably in a temperature range of 30 to 45° C., more preferably in a temperature range of 37 to 45° C., and even more preferably in a temperature range of 40 to 43° C. In the specification of the present application, “under isothermal conditions” means that the temperature is maintained in a temperature range of +3° C. or +1° C. relative to the temperature set during the reaction. The reaction time of homologous recombination is not particularly limited and may be, for example, 5 minutes to 6 hours, preferably 10 minutes to 2 hours, and even more preferably 15 minutes to 2 hours.

The recombinant circular DNA obtained by homologous recombination may have a gap or a nick. A gap is a state in which one or a plurality of continuous nucleotides are missing from double-stranded DNA, and a nick is a state in which a phosphate diester bond between adjacent nucleotides in the double-stranded DNA is cut. In the method for producing circular DNA according to the present invention, after homologous recombination, the gaps and nicks in the obtained recombinant circular DNA may be repaired by a gap repair enzyme group and dNTP. Repairing the gaps and nicks enables the conversion of the recombinant circular DNA into a complete double-stranded DNA.

Specifically, gap repair enzymes and dNTP are added to the reaction solution after homologous recombination and the reaction solution is incubated for a predetermined period of time under isothermal conditions at a temperature at which the gap repair enzymes are capable of exhibiting enzyme activity to repair the gaps and nicks of the recombinant circular DNA. The type or biological origin of the enzymes constituting the gap repair enzyme group are not particularly limited as long as the enzyme group is capable of repairing gaps and nicks in double-stranded DNA. For example, an enzyme having DNA polymerase activity and an enzyme having DNA ligase activity may be used in combination as the gap repair enzyme group. When a DNA ligase derived from Escherichia coli is used as the DNA ligase, NAD (nicotinamide adenine dinucleotide), which is a cofactor thereof, is contained in the reaction solution in a range of 0.01 to 1.0 mM. Treatment using the gap repair enzyme group may be performed, for example, at 25 to 40° C. for 5 to 120 minutes, and preferably 10 to 60 minutes.

dNTP is the generic name for dATP, dGTP, dCTP, and dTTP. The concentration of dNTP contained in the reaction solution at the start of the repair reaction may be in a range of, for example, 0.01 to 1 mM, and preferably in a range of 0.05 to 1 mM.

The recombinant circular DNA having repaired gaps and nicks is preferably also amplified. The method for amplifying the recombinant circular DNA having repaired gaps and nicks is not particularly limited, and amplification may generally be carried out by a method where linear or circular DNA is used as a template.

In one aspect of the method for producing circular DNA according to the present invention, it is preferable that the recombinant circular DNA obtained by further performing a reaction for repairing gaps and nicks after homologous recombination is amplified by a rolling circle amplification method (RCA). RCA may be performed by a conventional method.

In the method for producing circular DNA according to the present invention, when the recombinant circular DNA obtained by homologous recombination contains a replication origin (for example, origin of chromosome (oriC)) that is circular and is capable of binding to an enzyme having DnaA activity, the recombinant circular DNA is preferably amplified by a replication cycle reaction (RCR) amplification method. The recombinant circular DNA obtained by homologous recombination is directly obtained as is that is, RCR amplification is performed using the recombinant circular DNA as a template without performing a reaction for repairing gaps and nicks, thereby making it possible to obtain the recombinant circular DNA of complete double-stranded DNA without gaps and nicks as an amplification product.

A known replication origin present in bacteria such as Escherichia coli, Bacillus subtilis, and the like may be obtained as the replication origin from a public database such as NCBI. The replication origin may also be obtained by cloning a DNA fragment capable of binding to an enzyme having DnaA activity and analyzing the base sequence thereof. A modified sequence in which a mutation that substitutes, deletes, or inserts one or more bases from a known replication origin is inserted and that is capable of binding to an enzyme having DnaA activity may also be used as the replication origin used in the present invention. The replication origin used in the present invention is preferably oriC or a modified sequence thereof, and more preferably oriC derived from Escherichia coli or a modified sequence thereof.

Specifically, the RCR amplification method may be performed by forming a reaction mixture containing the recombinant circular DNA that is obtained by the homologous recombination and used as a template, a first enzyme group that catalyze replication of the circular DNA, a second enzyme group that catalyze ligation of Okazaki fragments so two sister circular DNA that form a catenane are synthesized, a third enzyme group that catalyze separation of the two sister circular DNA, and a dNTP and incubating the formed reaction mixture. The two sister circular DNA that form the catenane refers to two circular DNA synthesized by a DNA replication reaction being topologically connected.

A group of enzymes described in, for example, Kaguni JM & Kornberg A. Cell. 1984, 38:183-90 may be used as the first enzyme group that catalyze replication of the circular DNA. Specifically, examples of the first enzyme group may include one or more enzymes or groups of enzymes selected from the group composed of the following, or all of the combinations of these enzymes or groups of enzymes: enzymes having DnaA activity, one or more nucleoid proteins, enzymes or groups of enzymes having DNA gyrase activity, single-stranded DNA-binding proteins (single-stranded DNA-binding proteins (SSBs)), enzymes having DnaB-type helicase activity, enzymes having DNA helicase loader activity, enzymes having DNA primase activity, enzymes having DNA clamp activity, and enzymes or groups of enzymes having DNA polymerase III activity. In one aspect, the first enzyme group preferably includes an enzyme having DnaA activity, a single-stranded DNA binding protein (SSB), an enzyme having DnaB-type helicase activity, an enzyme having DNA helicase loader activity, an enzyme having DNA primase activity, an enzyme having DNA clamp activity, and an enzyme or a group of enzymes having DNA polymerase III activity.

The biological origin of the enzyme having DnaA activity is not particularly limited as long as it is an enzyme having the same initiator activity as DnaA, which is an initiator protein of Escherichia coli, but, for example, DnaA derived from Escherichia coli may be suitably used. DnaA derived from Escherichia coli may be contained in the reaction mixture as a monomer in a range of 1 nM to 10 μM, and preferably in a range of 1 nM to 5 μM, 1 nM to 3 μM, 1 nM to 1.5 μM, 1 nM to 1.0 μM, 1 to 500 nM, 50 to 200 nM, and 50 to 150 nM, but is not limited thereto.

The nucleoid protein is a protein contained in a nucleoid. The biological origin of the one or more nucleoid proteins used in the present invention is not particularly limited as long as the enzyme has the same activity as the nucleoid protein of Escherichia coli, but, for example, an IHF derived from Escherichia coli—that is, a complex of IhfA and/or IhfB (hetero dimer or homo dimer)—or HU derived from Escherichia coli—that is, a complex of hupA and hupB, may be suitably used. IHF derived from Escherichia coli may be contained in the reaction mixture as a hetero/homodimer in a range of 5 to 400 nM, and preferably in a range of 5 to 200 nM, 5 to 100 nM, 5 to 50 nM, 10 to 50 nM, 10 to 40 nM, and 10 to 30 nM, but is not limited thereto. HU derived from Escherichia coli may be contained in the reaction mixture in a range of 1 to 50 nM, and preferably in a range of 5 to 50 nM and 5 to 25 nM, but is not limited thereto.

The biological origin of the enzyme or the group of enzymes having DNA gyrase activity is not particularly limited as long as the enzyme has the same activity as DNA gyrase of Escherichia coli, but, for example, a complex composed of GyrA and GyrB derived from Escherichia coli may be suitably used. The complex composed of GyrA and GyrB derived from Escherichia coli may be contained in the reaction mixture as a heterotetramer in a range of 20 to 500 nM, and preferably in a range of 20 to 400 nM, 20 to 300 nM, 20 to 200 nM, 50 to 200 nM, and 100 to 200 nM, but is not limited thereto.

The biological origin of the SSB is not particularly limited as long as it is an enzyme having the same activity as a single-stranded DNA-binding protein of Escherichia coli, but, for example, an SSB derived from Escherichia coli may be suitably used. SSB derived from Escherichia coli may be contained in the reaction mixture as a homotetramer in a range of 20 to 1,000 nM, and preferably in a range of 20 to 500 nM, 20 to 300 nM, 20 to 200 nM, 50 to 500 nM, 50 to 400 nM, 50 to 300 nM, 50 to 200 nM, 50 to 150 nM, 100 to 500 nM, and 100 to 400 nM, but is not limited thereto.

The biological origin of the enzyme having DnaB-type helicase activity is not particularly limited as long as it is an enzyme having the same activity as DnaB of Escherichia coli, but, for example, DnaB derived from Escherichia coli may be suitably used. DnaB derived from Escherichia coli may be contained in the reaction mixture as a homohexamer in a range of 5 to 200 nM, and preferably in a range of 5 to 100 nM, 5 to 50 nM, and 5 to 30 nM, but is not limited thereto.

The biological origin of the enzyme having DNA helicase loader activity is not particularly limited as long as it is an enzyme having the same activity as DnaC of Escherichia coli, but, for example, DnaC derived from Escherichia coli may be suitably used. DnaC derived from Escherichia coli may be contained in the reaction mixture as a homohexamer in a range of 5 to 200 nM, and preferably in a range of 5 to 100 nM, 5 to 50 nM, and 5 to 30 nM, but is not limited thereto.

The biological origin of the enzyme having DNA primase activity is not particularly limited as long as it is an enzyme having the same activity as DnaG of Escherichia coli, but, for example, DnaG derived from Escherichia coli may be suitably used. DnaG derived from Escherichia coli may be contained in the reaction mixture as a monomer in a range of 20 to 1,000 nM, and preferably in a range of 20 to 800 nM, 50 to 800 nM, 100 to 800 nM, 200 to 800 nM, 250 to 800 nM, 250 to 500 nM, and 300 to 500 nM, but is not limited thereto.

The biological origin of the enzyme having DNA clamp activity is not particularly limited as long as it is an enzyme having the same activity as DnaN of Escherichia coli, but, for example, DnaN derived from Escherichia coli may be suitably used. DnaN derived from Escherichia coli may be contained in the reaction mixture as a homodimer in a range of 10 to 1,000 nM, and preferably in a range of 10 to 800 nM, 10 to 500 nM, 20 to 500 nM, 20 to 200 nM, 30 to 200 nM, and 30 to 100 nM, but is not limited thereto.

The biological origin of the enzyme or the group of enzymes having DNA polymerase III* activity is not particularly limited as long as it is an enzyme or a group of enzymes having the same activity as a DNA polymerase III* complex of Escherichia coli. For example, a group of enzymes containing any of DnaX, HolA, HolB, HolC, HolD, DnaE, DnaQ, and HolE derived from Escherichia coli, preferably a group of enzymes containing a complex of DnaX, HolA, HolB, and DnaE derived from Escherichia coli, and further preferably a group of enzymes containing a complex of DnaX, HolA, HolB, HolC, HolD, DnaE, DnaQ, and HolE derived from Escherichia coli may be suitably used. The DNA polymerase III* complex derived from Escherichia coli may be contained in the reaction mixture as a heteromultimer in a range of 2 to 50 nM, and preferably in a range of 2 to 40 nM, 2 to 30 nM, 2 to 20 nM, 5 to 40 nM, 5 to 30 nM, and 5 to 20 nM, but is not limited thereto.

Examples of the second enzyme group that catalyze ligation of Okazaki fragments so two sister circular DNA that form a catenane are synthesized include one or more enzymes selected from a group composed of enzymes having DNA polymerase I activity, enzymes having DNA ligase activity, and enzymes having RNaseH activity or a combination of these enzymes. In one aspect, the second enzyme group preferably includes enzymes having DNA polymerase I activity and enzymes having DNA ligase activity.

The biological origin of the enzyme having DNA polymerase I activity is not particularly limited as long as it is an enzyme having the same activity as DNA polymerase I of Escherichia coli, but, for example, DNA polymerase I derived from Escherichia coli may be suitably used. DNA polymerase I derived from Escherichia coli may be contained in the reaction mixture as a monomer in a range of 10 to 200 nM, and preferably in a range of 20 to 200 nM, 20 to 150 nM, 20 to 100 nM, 40 to 150 nM, 40 to 100 nM, and 40 to 80 nM, but is not limited thereto.

The biological origin of the enzyme having DNA ligase activity is not particularly limited as long as it is an enzyme having the same activity as DNA ligase of Escherichia coli, but, for example, DNA ligase derived from Escherichia coli and DNA ligase of T4 phage may be suitably used. DNA ligase derived from Escherichia coli may be contained in the reaction mixture as a monomer in a range of 10 to 200 nM, and preferably in a range of 15 to 200 nM, 20 to 200 nM, 20 to 150 nM, 20 to 100 nM, and 20 to 80 nM, but is not limited thereto.

The biological origin of the enzyme having RNaseH activity is not particularly limited as long as it has activity to degrade the RNA strand of an RNA: DNA hybrid, but, for example, RNaseH derived from Escherichia coli may be suitably used. RNaseH derived from Escherichia coli may be contained in the reaction mixture as a monomer in a range of 0.2 to 200 nM, and preferably in a range of 0.2 to 200 nM, 0.2 to 100 nM, 0.2 to 50 nM, 1 to 200 nM, 1 to 100 nM, 1 to 50 nM, and 10 to 50 nM, but is not limited thereto.

A group of enzymes described in, for example, Peng H & Marians KJ. PNAS. 1993, 90:8571-8575 may be used as the third enzyme group that catalyze separation of the two sister circular DNA. More specifically, examples of the third enzyme group may include one or more enzymes selected from the group composed of the following, or combinations of these enzymes: enzymes having topoisomerase IV activity, enzymes having topoisomerase III activity, and enzymes having RecQ-type helicase activity. In one aspect, the third enzyme group preferably includes enzymes having topoisomerase IV activity and/or enzymes having topoisomerase III activity.

The biological origin of the enzyme having topoisomerase III activity is not particularly limited as long as it is an enzyme having the same activity as topoisomerase III of Escherichia coli, but, for example, topoisomerase III derived from Escherichia coli may be suitably used. Topoisomerase III derived from Escherichia coli may be contained in the reaction mixture as a monomer in a range of 20 to 500 nM, and preferably in a range of 20 to 400 nM, 20 to 300 nM, 20 to 200 nM, 20 to 100 nM, and 30 to 80 nM, but is not limited thereto.

The biological origin of the enzyme having RecQ-type helicase activity is not particularly limited as long as it is an enzyme having the same activity as RecQ of Escherichia coli, but, for example, RecQ derived from Escherichia coli may be suitably used. RecQ derived from Escherichia coli may be contained in the reaction mixture as a monomer in a range of 20 to 500 nM, and preferably in a range of 20 to 400 nM, 20 to 300 nM, 20 to 200 nM, 20 to 100 nM, and 30 to 80 nM, but is not limited thereto.

The biological origin of the enzyme having topoisomerase IV activity is not particularly limited as long as it is an enzyme having the same activity as topoisomerase IV of Escherichia coli. For example, topoisomerase IV derived from Escherichia coli, which is a complex of ParC and ParE, may be suitably used. Topoisomerase IV derived from Escherichia coli may be contained in the reaction mixture as a heterotetramer in a range of 0.1 to 50 nM, and preferably in a range of 0.1 to 40 nM, 0.1 to 30 nM, 0.1 to 20 nM, 1 to 40 nM, 1 to 30 nM, 1 to 20 nM, 1 to 10 nM, and 1 to 5 nM, but is not limited thereto.

For the first, second, and third enzyme groups, those that are commercially available may be used, or they may be extracted from microorganisms and the like and purified as necessary. The extraction and purification of enzymes from microorganisms may be performed as appropriate using the techniques available to a person having ordinary skill in the art.

When enzymes other than those derived from Escherichia coli as described above are used as the first, second, and third enzyme groups, they may be used in a concentration range corresponding to the concentration range as an enzyme activity unit defined for the foregoing enzymes derived from Escherichia coli.

For the dNTP contained in the reaction mixture of RCR amplification, those that are given as examples for use in the method for producing circular DNA according to the present invention may be used.

The reaction mixture prepared in RCR amplification also contains, if necessary, a magnesium ion source, an alkali metal ion source, and ATP.

In RCR amplification, the concentration of ATP contained in the reaction mixture at the time the reaction starts may be in a range of, for example, 0.1 to 3 mM, and preferably in a range of 0.1 to 2 mM, 0.1 to 1.5 mM, and 0.5 to 1.5 mM.

The magnesium ion source contained in the reaction mixture may be used in RCR amplification in the same manner as that given as an example for use in the method for producing circular DNA according to the present invention. In RCR amplification, the concentration of the magnesium ion source contained in the reaction mixture at the start of the reaction may be, for example, a concentration at which magnesium ions are imparted in a range of 5 to 50 mM.

The alkali metal ion source contained in the reaction mixture may be used in RCR amplification in the same manner as that given as an example for use in the method for producing circular DNA according to the present invention. In RCR amplification, the concentration of the alkali metal ion source contained in the reaction mixture at the start of the reaction may be, for example, a concentration at which alkali metal ions are imparted at 100 mM or more, and preferably in a range of 100 to 300 mM, but is not limited to this.

In RCR amplification, the amount of recombinant circular DNA contained in the reaction mixture is not particularly limited. For example, at the start of the reaction, the recombinant circular DNA may be present in the reaction mixture at a concentration of 10 ng/μL or less, 5 ng/μL or less, 1 ng/μL or less, 0.8 ng/μL or less, 0.5 ng/μL or less, and 0.3 ng/μL or less.

By incubating the prepared reaction mixture under isothermal conditions at a predetermined temperature, only the circular DNA containing a replication origin capable of binding to an enzyme having DnaA activity is amplified. The reaction temperature in RCR amplification is not particularly limited as long as the DNA replication reaction is able to proceed, but may be in a range of, for example, 20 to 80° C., 25 to 50° C., or 25 to 40° C., which are the optimum temperatures for the DNA polymerase. The reaction time in RCR amplification may be set as appropriate according to the amount of amplification product of the desired recombinant circular DNA, but may be, for example, 30 minutes to 24 hours, and may be set to 24 hours or more.

RCR amplification may also be performed by incubating the prepared reaction mixture under a temperature cycle where incubation at 30° C. or higher and incubation at 27° C. or lower are repeated. The incubation at 30° C. or higher is not particularly limited as long as the temperature range allows the initiation of replication of circular DNA containing oriC, and may be, for example, 30 to 80° C., 30 to 50° C., 30 to 40° C., and 37° C. The incubation at 30° C. or higher is not particularly limited, but may be from 10 seconds to 10 minutes per cycle. The incubation at 27° C. or lower is not particularly limited as long as the temperature is such that replication initiation is suppressed and a DNA extension reaction progresses, and may be, for example, 10 to 27° C., 16 to 25° C., or 24° C. The incubation at 27° C. or lower is not particularly limited, but is preferably set to match the length of the recombinant circular DNA to be amplified and may be, for example, 1 to 10 seconds per 1,000 bases per cycle. The number of cycles for the temperature cycle is not particularly limited, but may be 10 to 50 cycles, 20 to 40 cycles, 25 to 35 cycles, or 30 cycles.

The recombinant circular DNA obtained by homologous recombination preferably undergoes heat treatment by incubation at 50 to 70° C. and then is rapidly cooled before being subjected to the gap and nick repair reaction or being used as a template for RCR amplification. The treatment time for heat treatment is not particularly limited and may be, for example, 1 to 15 minutes, and preferably 2 to 10 minutes. The temperature for rapid cooling is not particularly limited and, for example, it is cooled to 10° C. or less, and preferably 4° C. or less. The cooling rate during rapid cooling is preferably 50° C./min or more, more preferably 70° C./min or more, and even more preferably 85° C./min or more. For example, a container containing the heat-treated reaction mixture may be subjected to rapid cooling by directly placing the container on ice or bringing it into contact with a metal block regulated to 4° C. or less.

In the method for producing circular DNA according to the present invention, amplification of the recombinant circular DNA obtained by homologous recombination may be carried out by introducing the recombinant circular DNA into a microorganism and utilizing enzymes or the like in the microorganism. The recombinant circular DNA introduced into the microorganism may be recombinant circular DNA prior to the gap and nick repair reaction, or may be recombinant circular DNA following the repair reaction. Even when recombinant circular DNA having gaps and nicks is introduced into the microorganism as is, recombinant circular DNA in a state of being complete double-stranded DNA without gaps and nicks may be obtained as the amplification product. Examples of the microorganism in which the recombinant circular DNA is introduced include a microorganism having an enzyme capable of amplifying the circular DNA—for example, Escherichia coli, Bacillus subtilis, Actinomyces, Archaea, yeast, filamentous fungi, and the like. Introducing the recombinant circular DNA into the microorganism may be performed by a conventional method such as an electroporation method. The amplified recombinant circular DNA may also be recovered from the microorganism by a conventional method.

According to the present invention, the linear-DNA fragment may be incorporated into the circular DNA without cleaving the double-stranded circular DNA (that is, without linearizing both strands of the circular DNA); therefore, a circular DNA (for example, a plasmid) having the target gene introduced therein may be easily produced using a linear DNA fragment loaded with the target gene. For example, a plasmid having a drug resistance gene may be easily produced by using a linear-DNA fragment loaded with the drug resistance gene as the target gene and introducing the linear DNA fragment into the plasmid. In one aspect, the present invention relates to a method for producing double-stranded circular DNA having a target gene, wherein the linear-DNA fragment has the target gene between the homology region that corresponds to the region Ha and the homology region that corresponds to the region Hb, and linear circular DNA having the target gene is produced in which the region sandwiched between the region Ha and the region Hb in the circular double-stranded DNA is substituted with a region from the homologous region that corresponds to the region Ha to the homologous region that corresponds to the region Hb in the linear-DNA fragment.

Furthermore, the present invention also relates to a method for introducing a target gene into a double-stranded circular DNA, a method for introducing a drug resistance gene into a plasmid, and a method for producing a drug resistant plasmid. The plasmid is not particularly limited, but examples include well-known plasmids such as pUC, pBR322, pBluescript, pGEM, and pTZ plasmids. Well-known examples of drug resistance genes include ampicillin resistance genes, kanamycin resistance genes, and the like.

### <DNA-Fragment Recombining Kit>

By providing as a kit the proteins, reagents, and the like that are used in the method for producing circular DNA of the present invention, the method for producing circular DNA of the present invention can be carried out in a simpler manner, and circular DNA that is recombined with a linear-DNA fragment can be obtained. Specifically, a DNA-fragment recombining kit can be provided that includes, inter alia, a protein that has RecA-family-recombinase activity, the kit being used to produce circular DNA in which a region that is sandwiched by a region Ha and a region Hb in circular double-stranded DNA is substituted with the entirety or a portion of a linear-DNA fragment. The proteins that are listed above can be used as the protein that has RecA-family-recombinase activity.

The DNA-fragment recombining kit may include the pre-substituted circular DNA or the linear-DNA fragment (or both the pre-substituted circular DNA and the linear-DNA fragment) in addition to the protein that has RecA-family-recombinase activity. The pre-substituted circular DNA has the region Ha and the region Hb, which is located downstream of the region Ha. The linear-DNA fragment is single-stranded or double-stranded linear DNA that has a homologous region that corresponds to the region Ha in the pre-substituted circular DNA and a homologous region that corresponds to the region Hb in the pre-substituted circular DNA. In one aspect, the linear-DNA fragment that is included in the DNA-fragment recombining kit is a fragment in which a portion of a double-stranded DNA fragment is made into a single strand. As described above, the fragment in which a portion thereof is made into a single strand can be preprepared by using a USER (registered trademark) enzyme or the like or by ligating single-stranded DNA together that have different lengths and/or complementary regions.

The DNA-fragment recombining kit preferably further includes an exonuclease when the linear-DNA fragment is a linear double-stranded DNA fragment and the homologous region that corresponds to the region Ha and/or the homologous region that corresponds to the region Hb is double-stranded DNA. The RecA-family recombinase protein and the exonuclease that are provided in the DNA-fragment recombining kit are added to a solution that contains the pre-substituted circular DNA and linear-DNA fragment that are to be recombined. The addition enables the method for producing circular DNA of the present invention to be performed in a simpler manner and enables the recombinant circular DNA that is being sought to be obtained more easily. The RecA-family recombinase protein and the exonuclease that are used in the method for producing circular DNA of the present invention can be used as-is as the RecA-family recombinase protein and the exonuclease that are included in the DNA-fragment recombining kit. The exonuclease that is included in the DNA-fragment recombining kit may be a 3′ to 5′ exonuclease or a 5′ to 3′ exonuclease. An exonuclease that is specific to linear double-stranded DNA is preferably included as the exonuclease that is included in the DNA-fragment recombining kit.

The DNA-fragment recombining kit preferably further includes an enzyme for regenerating a nucleoside triphosphate or deoxynucleotide triphosphate and preferably further includes a substrate for the enzyme. The DNA-fragment recombining kit can also include one or more types of substances that are selected from the group consisting of a nucleoside triphosphate, a deoxynucleotide triphosphate, a magnesium-ion source, an alkali-metal-ion source, dimethyl sulfoxide, tetramethylammonium chloride, polyethylene glycol, dithiothreitol, and a buffer. The components that are used in the method for producing circular DNA of the present invention can be used as-is as the above components.

The DNA-fragment recombining kit preferably further includes a document describing the protocols for using the DNA-fragment recombining kit to carry out the method for producing circular DNA of the present invention. The protocols may be described on the surface of a container that houses the DNA-fragment recombining kit.

### EXAMPLES

Next, the present invention is described in greater detail by means of examples and the like. However, the present invention is not limited by the examples and the like.

### Example 1

A linear double-stranded DNA fragment was inserted into circular double-stranded DNA by homologous recombination by using a RecA-family recombinase protein and a 3′ to 5′ exonuclease.

A wild type of Escherichia coli RecA (patent literature 4) was used as the RecA-family recombinase protein, and exonuclease III was used as the 3′ to 5′ exonuclease. The plasmid pUC4K (GenBank accession no.: X06404, total length of 3.9 kbp) was used as the circular double-stranded DNA, the region that consists of SEQ ID NO: 1 in the plasmid was made to be the region Ha (40 bp), and the region that consists of SEQ ID NO: 2 and is located downstream of and adjacent to the region Ha was made to be the region Hb (40 bp). A linear double-stranded DNA fragment (oriC_pUCori1 cassette) that has a base sequence (SEQ ID NO: 3) in which the oriC sequence in pUC is ligated downstream of a base sequence that is identical to the region Ha and in which a base sequence that is identical to the region Hb is ligated downstream of the oriC sequence was used as the linear double-stranded DNA fragment. The base sequence of the oriC_pUCori1 cassette is shown in table 1. In the base sequence of SEQ ID NO: 3 in table 1, the upstream lowercase region (5′ end side) is the base sequence that is identical to the region Ha, and the downstream lowercase region (3′ end side) is the base sequence that is identical to the region Hb.

TABLE 1

| | | SEQ ID |
|---|---|---|
| | Base sequence | NO: |
| oriC_pUCori1 | gggtctgacgctcagtggaacgaaaactcacgttaagggaGCGGCCGCGAAGATCCGGC | 3 |
| cassette | AGAAGAATGGAGTATGTTGTAACTAAAGATAACTTCGTATAATGTATGCTATACGAAGT | |
| | TATACAGATCGTGCGATCTACTGTGGATAACTCTGTCAGGAAGCTTGGATCAACCGGTA | |
| | GTTATCCAAAGAACAACTGTTGTTCAGTTTTTGAGTTGTGTATAACCCCTCATTCTGAT | |
| | CCCAGCTTATACGGTCCAGGATCACCGATCATTCACAGTTAATGATCCTTTCCAGGTTG | |
| | TTGATCTTAAAAGCCGGATCCTTGTTATCCACAGGGCAGTGCGATCCTAATAAGAGATC | |
| | ACAATAGAACAGATCTCTAAATAAATAGATCTTCTTTTTAATACCCAGGATCCCTTTAG | |
| | TTACAACATACTCAGGTCTTTCTCAAGCCGAttttggtcatgagattatcaaaaaggat | |
| | cttcacctagat | |

For the homologous recombination, 40 pM of pUC4K and 40 pM of the oriC_pUCori1 cassette were added to 5 μL of a homologous-recombination solution (RM solution) (1 μM of RecA, 80 mU/μL of exonuclease III, 20 mM of Tris-HCl (pH 8.0), 4 mM of DTT, 1 mM of magnesium acetate, 100 μM of ATP, 4 mM of creatine phosphate, 20 ng/μL of creatine kinase, 50 mM of potassium glutamate, 150 mM of TMAC, 5 mass % of PEG 8000, and 10 vol % of DMSO), and the reaction solution was incubated for 30 minutes at 37° C. An RCR-amplification reaction solution (5 μL) was prepared by mixing 0.5 μL of the reacted reaction solution with 4.5 μL of a liquid mixture in which 60 nM of Tus is contained in the reaction mixture of the composition that is shown in table 2. RCR amplification was performed by incubating the RCR-amplification reaction solution for 16 hours at 30° C. The Tus was prepared from Escherichia coli strain that expresses Tus, the preparation involving purification by a process that includes affinity column chromatography and gel permeation column chromatography.

TABLE 2
Reaction mixture
Reaction buffer

| | | |
|---|---|---|
| Tris-HCl (pH 8.0) | 20 | mM |
| Dithiothreitol | 8 | mM |
| Potassium glutamate | 150 | mM |
| Mg (OAc)₂ | 10 | mM |
| Creatine phosphate | 4 | mM |
| ATP | 1 | mM |
| GTP, CTP, UTP | 1 | mM each |
| dNTPs | 0.1 | mM each |
| tRNA | 50 | ng/μL |
| NAD | 0.25 | mM |
| Ammonium sulfate | 10 | mM |
| Bovine serum albumin (BSA) | 0.5 | mg/mL |
| Creatine kinase | 20 | ng/μL |

Enzyme group

| | | |
|---|---|---|
| SSB | 400 | nM |
| IHF | 20 | nM |
| DnaG | 400 | nM |
| DnaN | 40 | nM |
| Pol III* | 5 | nM |
| DnaB, DnaC | 20 | nM |
| DnaA | 100 | nM |
| RNaseH | 10 | nM |
| Ligase | 50 | nM |
| Pol I | 50 | nM |
| GyrA, GyrB | 50 | nM |
| Topo IV | 5 | nM |
| Topo III | 50 | nM |
| RecQ | 50 | nM |

In table 2, “SSB” represents SSB that is derived from Escherichia coli; “IHF” represents a complex of IhfA and IhfB that are derived from Escherichia coli; “DnaG” represents DnaG that is derived from Escherichia coli; “DnaN” represents DnaN that is derived from Escherichia coli; “Pol III*” represents a DNA polymerase III* complex that is composed of DnaX, HolA, HolB, HolC, HolD, DnaE, DnaQ, and HolE that are derived from Escherichia coli; “DnaB” represents DnaB that is derived from Escherichia coli; “DnaC” represents Dnac that is derived from Escherichia coli; “DnaA” represents DnaA that is derived from Escherichia coli; “RNaseH” represents RNaseH that is derived from Escherichia coli; “Ligase” represents a DNA ligase that is derived from Escherichia coli; “Pol I” represents DNA polymerase I that is derived from Escherichia coli; “GyrA” represents GyrA that is derived from Escherichia coli; “GyrB” represents GyrB that is derived from Escherichia coli; “Topo IV” represents a complex of ParC and ParE that are derived from Escherichia coli; “Topo III” represents topoisomerase III that is derived from Escherichia coli; and “RecQ” represents RecQ that is derived from Escherichia coli.

The SSB was prepared from Escherichia coli strain that expresses SSB, the preparation involving purification by a process that includes ammonium sulfate precipitation and ion-exchange column chromatography.

The IHF was prepared from Escherichia coli strain that co-expresses IhfA and IfhB, the preparation involving purification by a process that includes ammonium sulfate precipitation and affinity column chromatography.

The DnaG was prepared from Escherichia coli strain that expresses DnaG, the preparation involving purification by a process that includes ammonium sulfate precipitation, anion-exchange column chromatography, and gel permeation column chromatography.

The DnaN was prepared from Escherichia coli strain that expresses DnaN, the preparation involving purification by a process that includes ammonium sulfate precipitation and anion-exchange column chromatography.

The Pol III* was prepared from Escherichia coli strain that co-expresses DnaX, HolA, HolB, HolC, HolD, DnaE, DnaQ, and HolE, the preparation involving purification by a process that includes ammonium sulfate precipitation, affinity column chromatography, and gel permeation column chromatography.

The DnaB and DnaC were prepared from Escherichia coli strain that co-expresses DnaB and DnaC, the preparation involving purification by a process that includes ammonium sulfate precipitation, affinity column chromatography, and gel permeation column chromatography.

The DnaA was prepared from Escherichia coli strain that expresses DnaA, the preparation involving purification by a process that includes ammonium sulfate precipitation, dialysis precipitation, and gel permeation column chromatography.

The GyrA and GyrB were prepared from a mixture of Escherichia coli strain that expresses GyrA and Escherichia coli strain that expresses GyrB, the preparation involving purification by a process that includes ammonium sulfate precipitation, affinity column chromatography, and gel permeation column chromatography.

The Topo IV was prepared from a mixture of Escherichia coli strain that expresses ParC and Escherichia coli strain that expresses ParE, the preparation involving purification by a process that includes ammonium sulfate precipitation, affinity column chromatography, and gel permeation column chromatography.

The Topo III was prepared from Escherichia coli strain that expresses Topo III, the preparation involving purification by a process that includes ammonium sulfate precipitation and affinity column chromatography.

The RecQ was prepared from Escherichia coli strain expresses RecQ, the preparation involving purification by a process that includes ammonium sulfate precipitation, affinity column chromatography, and gel permeation column chromatography.

Commercially available enzymes (manufactured by Takara Bio) that are derived from Escherichia coli were used as the RnaseH, Ligase, and Pol I.

After the end of the RCR amplification, a portion (0.4 μL) of the RCR-amplification reaction solution was diluted to one-tenth in an RCR buffer (“Reaction buffer” in the composition shown in table 2). The diluted solution was then incubated for 30 minutes at 30° C. Afterward, 1 μL of the diluted solution was subjected to agarose electrophoresis, and the separated bands were stained by using SYBR (registered trademark) Green.

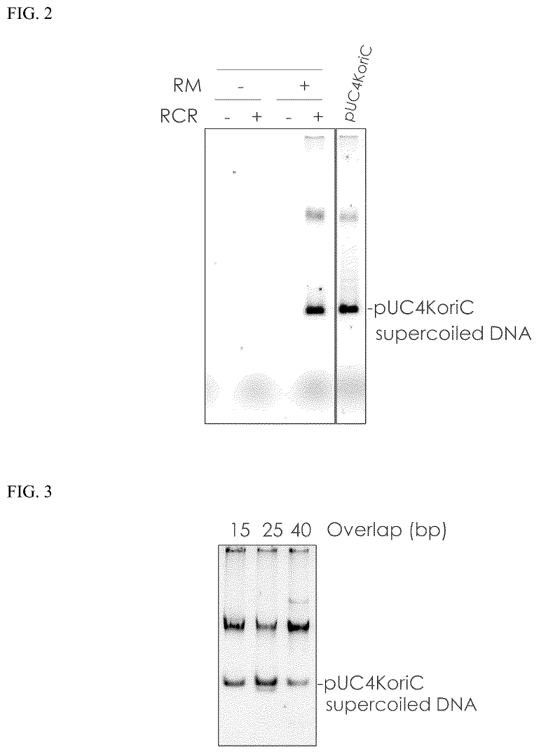

The staining results are shown in FIG. 2. In the figure, the lane in which “RM” is “−” is a lane where a solution in which the oriC_pUCori1 cassette and pUC4K are dissolved in water, the solution being incubated for 30 minutes at 37° C., was subjected to electrophoresis, and the lane in which “RM” is “+” is a lane where the solution in which the oriC_pUCori1 cassette and pUC4K are dissolved in the RM solution, the solution being subjected to homologous recombination, was subjected to electrophoresis. The lane in which “RCR” is “−” is a lane where 1 μL of a dilution in which the homologously recombined reaction solution is diluted to one-hundredth by means of an RCR buffer is subjected to electrophoresis, and the lane in which “RCR” is “+” is a lane where a solution in which the homologously recombined reaction solution is subjected to RCR amplification, the obtained reaction solution is afterward diluted by means of an RCR buffer, and the dilution is incubated is subjected to electrophoresis. As the control, a product (pUC4KoriC) that was subjected to RCR amplification after the ligation of a 3.9 kb PCR fragment, in which the region Ha and the region Hb are present at the ends thereof, and the oriC_pUCori1 cassette according to the recombination assembly method disclosed in patent literature 4, pUC4K being used as the template, was also subjected to electrophoresis.

As shown in FIG. 2, a band was not detected in the solution that was not subjected to homologous recombination (the lane where “RM” is “−”), irrespective of the presence or absence of RCR amplification. In contrast, a band of the same size as the band of a supercoil of pUC4KoriC was confirmed in the homologously recombined solution (the lane where “RM” is “+”) that was subjected to RCR amplification (the lane where “RCR” is “+”). It was confirmed from the results that by incubating, in the RM solution, pUC4K that is still circular and the oriC_pUCori1 cassette gives rise to homologous recombination and oriC_pUCori1 is inserted into pUC4K.

### Example 2

The effect of the length, in number of bases, of the homologous regions (region Ha, region Hb) was examined by using linear double-stranded DNA fragments in which the region Ha and the region Hb are 15 bp, 25 bp, or 40 bp in homologous recombination in which the linear double-stranded DNA fragment is inserted into circular double-stranded DNA by using a RecA-family recombinase protein and a 3′ to 5′ exonuclease.

An oriC_ColE1 cassette in which the homologous regions (region Ha, region Hb) are added to the ends of a linear double-stranded DNA fragment that contains oriC (oriC2.0) (SEQ ID NO: 4) was used as the linear double-stranded DNA fragment. PCR was performed by using oriC2.0 as the template, using a forward primer of the base sequence represented by SEQ ID NO: 5, and using a reverse primer of the base sequence that is given at SEQ ID NO: 6, and the obtained amplification product was used as a linear double-stranded DNA fragment in which the region Ha and the region Hb are 15 bp (15 bp overlap cassette). Similarly, a PCR product that was obtained from a forward primer of the base sequence represented by SEQ ID NO: 7 and a reverse primer of the base sequence represented by SEQ ID NO: 8, oriC2.0 being used as the template, was used as a linear double-stranded DNA fragment in which the region Ha and the region Hb are 25 bp (25 bp overlap cassette), and a PCR product that was obtained from a forward primer of the base sequence represented by SEQ ID NO: 9 and a reverse primer of the base sequence represented by SEQ ID NO: 10, oriC2.0 being used as the template, was used as a linear double-stranded DNA fragment in which the region Ha and the region Hb are 40 bp (40 bp overlap cassette). In the base sequence of SEQ ID NO: 4, the underlined portions indicate the regions that hybridize with the primers. In the primers, the lowercase regions indicate the homologous regions (region Ha or region Hb).

TABLE 3

| | | SEQ ID |
|---|---|---|
| | Base sequence | NO: |
| oriC2.0 | GAGCTGTTGACAGAGGGTCATTTTCACACTATAATGCAGTGAATCCCAAACAGTATGTT | 4 |
| | GTACCTAAAGGGATCCTGGGTATTAAAAAGAAGATCTATTTATTTAGAGATCTGTTCTA | |
| | TTGTGATCTCTTATTAGGATCGCACTGCCCTGTGGATAACAAGGATCCGGCTTTTAAGA | |
| | TCAACAACCTGGAAAGGATCATTAACTGTGAATGATCGGTGATCCTGGACCGTATAAGC | |
| | TGGGATCAGAATGAGGGGTTATACACAACTCAAAAACTGAACAACAGTTGTTCTTTGGA | |
| | TAACTACCGGTTGATCCAAGCTTCCTGACAGAGTTATCCACAGTAGATCGCACGATCTG | |
| | TCAGCTCATTTCCTTTAGGTACAACATACTAGAATATTTGCCTACAGCCTCCTTTCGGA | |
| | GGCTGTTTTTTTA | |
| 15 bp_F primer | accccgtagaaaagaGAGCTGTTGACAGAGGGTC | 5 |
| 15 bp_R primer | aagaagatcctttgaTAAAAAAACAGCCTCCGAAAGGAG | 6 |
| 25 bp_F primer | tgagcgtcagaccccgtagaaaagaGAGCTGTTGACAGAGGGTG | 7 |
| 25 bp_R primer | aaaggatctcaagaagatcctttgaTAAAAAAACAGCCTCCGAAAGGAG | 8 |
| 40 bp_F primer | gagttttcgttccactgagcgtcagaccccgtagaaaagaGAGCTGTTGACAGAGGGTC | 9 |
| 40 bp_R primer | attacgcgcagaaaaaaaggatctcaagaagatcctttgaTAAAAAAACAGCCTCCGAA | 10 |
| | AGGAG | |

Specifically, homologous recombination and RCR amplification were performed in the same manner as Example 1 other than making the oriC_ColE1 cassette and pUC4K that are added to the reaction solution in the homologous recombination be 800 pM. The obtained amplification product was subjected to electrophoresis, and the bands were stained.

The staining results are shown in FIG. 3. In the figure, the lanes in which “Overlap (bp)” is “15,” “25,” and “40” are lanes where the RCR-amplification reaction products of the reaction solutions in which the 15 bp overlap cassette, the 25 bp overlap cassette, and the 40 bp overlap cassette are used were subjected to electrophoresis. As shown in FIG. 3, homologous recombination was confirmed to occur such that the oriC_ColE1 cassette is inserted into pUC4K, regardless of whether the homologous region is 15 bp, 25 bp, or 40 bp.

### Example 3

The effect of the reaction temperature of homologous recombination in homologous recombination in which a linear double-stranded DNA fragment is inserted into circular double-stranded DNA by using a RecA-family recombinase protein and a 3′ to 5′ exonuclease was examined.

Homologous recombination and RCR amplification were performed in the same manner as Example 1 other than using an oriC_lac cassette (SEQ ID NO: 11) that contains oriC, the 40 bp at each end of the cassette being sequences that are homologous to the adjacent 40 bp regions of pUC4K, as the linear double-stranded DNA fragment and performing the homologous recombination at 24° C., 30° C., 37° C., or 42° C. The obtained amplification product was subjected to electrophoresis, and the bands were stained. In the base sequence of SEQ ID NO: 11, the upstream lowercase region (5′ end side) is the base sequence that is identical to the region Ha, and the downstream lowercase region (3′ end side) is the base sequence that is identical to the region Hb. The region Ha and the region Hb are the adjacent 40 bp regions of pUC4K.

TABLE 4

| | | SEQ ID |
|---|---|---|
| | Base sequence | NO: |
| oriC_lac | atggtgcactctcagtacaatctgctctgatgccgcatAGTATGTTGTAACTAAAGATCTA | 11 |
| cassette | CTGTGGATAACTCTGTCAGGAAGCTTGGATCAACCGGTAGTTATCCAAAGAACAACTGTTG | |
| | TTCAGTTTTTGAGTTGTGTATAACCCCTCATTCTGATCCCAGCTTATACGGTCCAGGATCA | |
| | CCGATCATTCACAGTTAATGATCCTTTCCAGGTTGTTGATCTTAAAAGCCGGATCCTTGTT | |
| | ATCCACAGGGGAGTGCGATCCTAATAAGAGATCACAATAGAACAGATCTCTAAATAAATAG | |
| | ATCTTCTTTTTAATACTTTAGTTACAACATACTgttaagccagccccgacacccgccaaca | |
| | cccgctgacgcg | |

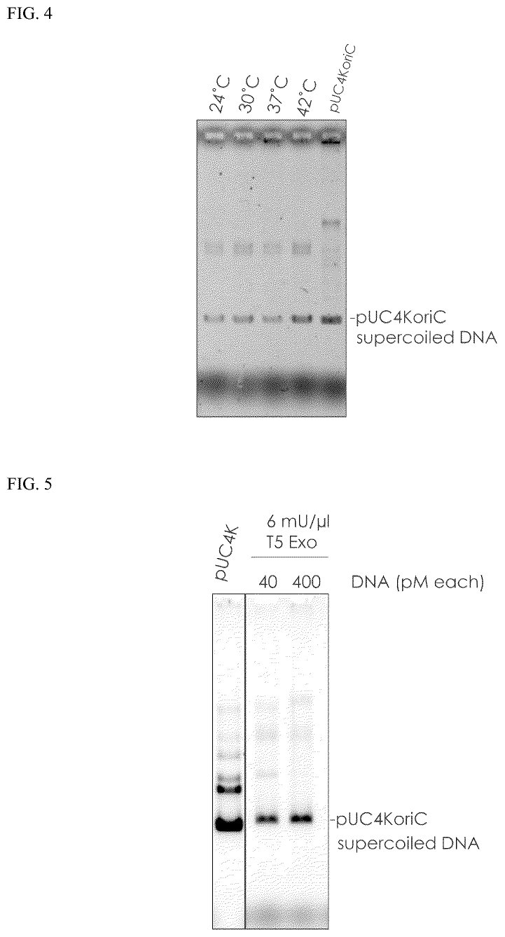

The staining results are shown in FIG. 4. In the figure, the lanes “24° C.,” “30° C.,” “37° C.,” and “42° C.” are lanes where the RCR-amplification reaction products of reaction solutions in which the reaction temperature of the homologous recombination was respectively 24° C., 30° C., 37° C., and 42° C. were subjected to electrophoresis. As shown in FIG. 4, homologous recombination was confirmed to occur such that the oriC_lac cassette is inserted into pUC4K, regardless of whether the reaction temperature of the homologous recombination is 24° C., 30° C., 37° C., or 42° C.

### Example 4

A linear double-stranded DNA fragment was inserted into circular double-stranded DNA by homologous recombination by using a RecA-family recombinase protein and a 5′ to 3′ exonuclease. T5 exonuclease was used as the 5′ to 3′ exonuclease. The oriC_lac cassette that was used in Example 3 was used as the linear double-stranded DNA fragment.

Homologous recombination and RCR amplification were performed in the same manner as Example 1 other than adding 6 mU/μL of T5 exonuclease instead of 80 mU/μL of exonuclease III to the RM solution and adding 40 pM of the oriC_lac cassette and 40 pM of pUC4K, or 400 pM of the oriC_lac cassette and 400 pM of pUC4K, to the homologous-recombination reaction solution. The obtained amplification product was subjected to electrophoresis, and the bands were stained.

The staining results are shown in FIG. 5. In the figure, the lanes in which “DNA” is “40” and “400” are lanes where the RCR-amplification reaction product of the reaction solution where the oriC_lac cassette and pUC4K are added at 40 pM or 400 pM each to the homologous-recombination reaction solution were subjected to electrophoresis. As shown in FIG. 5, even when a 5′ to 3′ exonuclease was used, homologous recombination occurred such that the oriC_lac cassette is inserted into pUC4K in the same manner as Example 1, etc., which use a 3′ to 5′ exonuclease.

### Example 5

The effect of the concentration of a RecA-family-recombinase protein in the reaction solution in homologous recombination in which a linear double-stranded DNA fragment is inserted into circular double-stranded DNA by using the RecA-family recombinase protein and a 5′ to 3′ exonuclease was examined.

The plasmid pBeloBAC11 (GenBank accession no.: U51113, total length of 7.5 kbp) was used as the circular double-stranded DNA, and 60 bp regions that are adjacent to each other were set to be the region Ha and the region Hb. A linear double-stranded DNA fragment (oriC_sopC cassette) that has a base sequence (SEQ ID NO: 12) in which the oriC sequence is ligated downstream of a base sequence that is identical to the region Ha and in which a base sequence that is identical to the region Hb is ligated downstream of the oriC sequence was used as the linear double-stranded DNA fragment. The base sequence of the oriC_sopC cassette is shown in table 3. In the base sequence of SEQ ID NO: 12 in table 3, the upstream lowercase region (5′ end side) is the base sequence that is identical to the region Ha, and the downstream lowercase region (3′ end side) is the base sequence that is identical to the region Hb.

TABLE 5

| | | SEQ ID |
|---|---|---|
| | Base sequence | NO: |
| oriC_sopC | ggtcccacttgtattgtcgatcagactatcagcgtgagactacgattccatcaatgcctgG | 12 |
| cassette | AAGATCCGGCAGAAGAATGGAGTATGTTGTAACTAAAGATAACTTCGTATAATGTATGCTA | |
| | TACGAAGTTATACAGATCGTGCGATCTAGTGTGGATAACTGTGTCAGGAAGCTTGGATCAA | |
| | CCGGTAGTTATCCAAAGAACAACTGTTGTTCAGTTTTTGAGTTGTGTATAACCCCTCATTC | |
| | TGATCCCAGCTTATACGGTCCAGGATCACCGATCATTCACAGTTAATGATCCTTTCCAGGT | |
| | TGTTGATCTTAAAAGCCGGATCCTIGTTATCCACAGGGCAGTGCGATCCTAATAAGAGATC | |
| | ACAATAGAACAGATCTCTAAATAAATAGATCTTCTTTTTAATACCCAGGATCCATCTATGT | |
| | CGGGTGCGGAGAAAGAGGTAATGAAATGGCTTTAGTTACAACATACTCAGGTCTTTCTCAA | |
| | GCCGAtcaagggcaagtattgacatgtcgtcgtaacctgtagaacggagtaacctcggtgt | |
| | gcgg | |

Homologous recombination and RCR amplification were performed in the same manner as Example 4 other than setting the concentration of the RecA-family-recombinase protein that is added to the RM solution to be 0, 0.1, 0.3, 1, or 3 UM and adding the oriC_sopC cassette and pBeloBAC11 instead of the pUC4K and the oriC_pUCori1 cassette that were added to the homologous-recombination reaction solution. The obtained amplification product was subjected to electrophoresis, and the bands were stained.

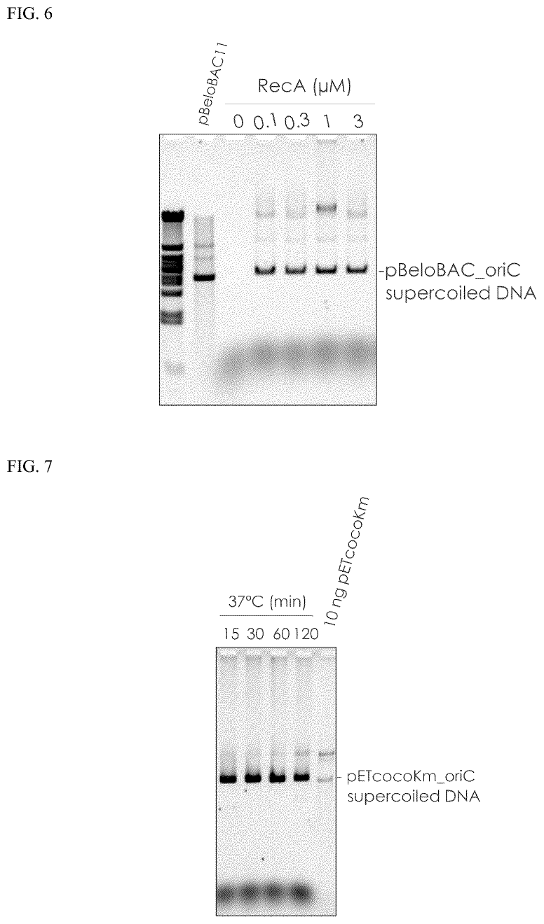

The staining results are shown in FIG. 6. In the figure, the lanes in which “RecA” is “0,” “0.1,” “0.3,” “1,” and “3” are lanes where the RCR-amplification reaction product of the reaction solution in which the concentration of the RecA-family-recombinase protein in the RM solution that is used for the homologous recombination is set to 0, 0.1, 0.3, 1, or 3 μM was subjected to electrophoresis. “pBeloBAC11” is the lane where 10 ng of pBeloBAC11 was subjected to electrophoresis. As shown in FIG. 6, a band where the oriC_sop cassette is inserted into pBeloBAC11 was confirmed as the amplification product only when the homologous recombination was performed in the presence of a RecA-family-recombinase protein.

### Example 6

The effect of the reaction time in homologous recombination in which a linear double-stranded DNA fragment is inserted into circular double-stranded DNA by using a RecA-family recombinase protein and a 5′ to 3′ exonuclease was examined. The plasmid pETcoco (registered trademark) Km (non-patent literature 3, total length of 11.3 kbp) was used as the circular double-stranded DNA, and the oriC_sopC cassette that was used in Example 5 was used as the linear double-stranded DNA fragment.

Homologous recombination and RCR amplification were performed in the same manner as Example 4 other than adding the oriC_sopC cassette and pETcocoKm instead of the pUC4K and the oriC_pUCori1 cassette that were added to the homologous-recombination reaction solution and setting the reaction time of the homologous recombination to 15, 30, 60, or 120 minutes. The obtained amplification product was subjected to electrophoresis, and the bands were stained.

The staining results are shown in FIG. 7. As shown in the figure, homologous recombination occurred such that the oriC_sopC cassette is inserted into pETcocoKm, regardless of the length of the reaction time of the homologous recombination.

### Example 7

In homologous recombination in which a linear double-stranded DNA fragment is inserted into circular double-stranded DNA by using a RecA-family-recombinase protein and a 3′ to 5′ exonuclease, the region from the region Ha to the region Hb in the circular double-stranded DNA was substituted with the linear double-stranded DNA fragment by homologous recombination.

pUC4K was used as the circular double-stranded DNA. The oriC_lac cassette that was used in Example 3, or an oriC_668 cassette of a base sequence (SEQ ID NO: 13) in which the oriC sequence in pUC is ligated downstream of a base sequence that is identical to the region Ha and in which a base sequence that is identical to the region Hb is ligated downstream of the oriC sequence, was used as the linear double-stranded DNA fragment. In the base sequence of SEQ ID NO: 13 in table 6, the upstream lowercase region (40 bp, 5′ end side) is the base sequence that is identical to the region Ha, and the downstream lowercase region (40 bp, 3′ end side) is the base sequence that is identical to the region Hb. The region Ha and the region Hb in the oriC_668 cassette are 668 bp apart in pUC4K and thus differ from the region Ha and the region Hb of the oriC_lac cassette, which are designed to be adjacent to each other.

TABLE 6

| | | SEQ ID |
|---|---|---|
| | Base sequence | NO: |
| oriC_668 | gagcaaaaggccagcaaaaggccaggaaccgtaaaaaggcAGTATGTTGTAACTAAAGATC | 13 |
| cassette | TACTGTGGATAACTCTGTCAGGAAGCTTGGATCAACCGGTAGTTATCCAAAGAACAACTGT | |
| | TGTTCAGTTTTTGAGTTGTGTATAACCCCTCATTCTGATCCCAGCTTATACGGTCCAGGAT | |
| | CACCGATCATTCACAGTTAATGATCCTTTCCAGGTTGTTGATCTTAAAAGCCGGATCCTTG | |
| | TTATCCACAGGGCAGTGCGATCCTAATAAGAGATCACAATAGAACAGATCTCTAAATAAAT | |
| | AGATCTTCTTTTTAATACTTTAGTTACAACATACTttttggtcatgagattatcaaaaagg | |
| | atcttcacctagat | |

Homologous recombination and RCR amplification were performed in the same manner as Example 1 other than adding 80 pM of pUC4K and 80 pM of the oriC_lac cassette, or 400 PM of pUC4K and 400 pM of the oriC_668 cassette, to the homologous-recombination reaction solution. After the end of the RCR amplification, a portion (0.4 μL) of the RCR-amplification reaction solution was diluted to one-tenth in an RCR buffer. The diluted solution was then incubated for 30 minutes at 30° C. Afterward, 1 μL of the diluted solution was subjected as-is to agarose electrophoresis, and the separated bands were stained by using SYBR Green. 1 μL of the diluted solution was added to an enzyme solution that contains the restriction enzyme EcoRI. The mixture was digested by incubating the mixture for 30 minutes at 37° C. Afterward, the digested mixture was subjected to agarose electrophoresis, and the separated bands were stained by using SYBR Green.

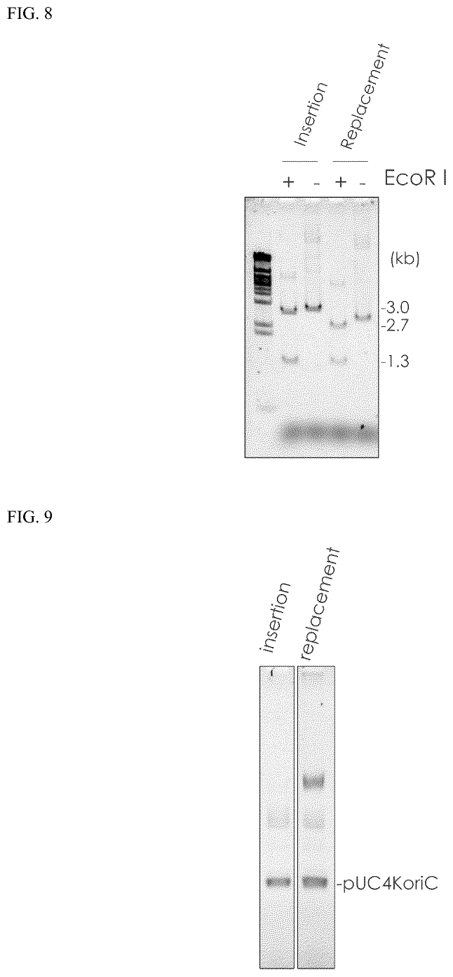

The staining results are shown in FIG. 8. In the figure, the “Insertion” lanes are lanes where the homologous-recombination reaction solution of pUC4K and the oriC_lac cassette is subjected to electrophoresis, and the “Replacement” lanes are lanes where the homologous-recombination reaction solution of pUC4K and the oriC_668 cassette is subjected to electrophoresis. The lanes in which “EcoRI” is “−” are lanes where a reaction solution that is not subjected to the EcoRI digestion process is subjected to electrophoresis, and the lanes in which “EcoRI” is “+” are lanes where a reaction solution that is subjected to the EcoRI digestion process is subjected to electrophoresis.

When homologous recombination is performed by using pUC4K and the oriC_lac cassette, circular DNA in which 300 bp from the oriC_lac cassette are inserted between the region Ha and the region Hb of pUC4K—that is, circular DNA that is 300 bp longer than the pre-reaction pUC4K—is obtained. When homologous recombination is performed by using pUC4K and the oriC_668 cassette, circular DNA in which a 668 bp region that is sandwiched by the region Ha and the region Hb in pUC4K is substituted with 300 bp from the oriC_668 cassette—that is, circular DNA that is 300 bp shorter than the pre-reaction pUC4K—is obtained. In fact, as shown in FIG. 8, with regard to a digested product that is an RCR amplification product that is made into a single strand by EcoRI digestion, the bands of the digested product of the circular DNA that was obtained by homologous recombination of pUC4K and the oriC_lac cassette were clearly longer than the bands of the digested product of the circular DNA that was obtained by homologous recombination of pUC4K and the oriC_668 cassette. It was confirmed from the results that circular DNA of the predicted lengths are obtained by homologous recombination.

### Example 8

In homologous recombination in which a linear double-stranded DNA fragment is inserted into circular double-stranded DNA by using a RecA-family-recombinase protein and a 3′ to 5′ exonuclease, the region from the region Ha to the region Hb in the circular double-stranded DNA was substituted with the linear double-stranded DNA fragment by homologous recombination.

pUC4K was used as the circular double-stranded DNA. An oriC_pUCori2 cassette (SEQ ID NO: 14) that has the oriC sequence or an oriC_1760 cassette (SEQ ID NO: 15) that has a 0.9 kb lambda-phage-DNA-derived base sequence upstream and downstream of the oriC sequence was used as the linear double-stranded DNA fragment. In the base sequences of SEQ ID NO: 14 and SEQ ID NO: 15 in table 7, the upstream lowercase region (5′ end side) is the base sequence that is identical to the region Ha, and the downstream lowercase region (3′ end side) is the base sequence that is identical to the region Hb. The region Ha and region Hb (40 bp) that are set in the oriC_pUCori2 cassette were designed to be in positions that are adjacent to each other in pUC4K. The region Ha and region Hb (120 bp) that are set in the oriC_1760 cassette were designed to be in positions that are separated by 1760 bp in pUC4K.

TABLE 7

| | | SEQ ID |
|---|---|---|
| | Base sequence | NO: |
| oriC_ | gggtctgacgctcagtggaacgaaaactcacgttaagggaAGTATGTTGTAACTAAAGATCTACTGTG | 14 |
| pUCori2 | GATAACTCTGTCAGGAAGCTTGGATCAACCGGTAGTTATCCAAAGAACAACTGTTGTTCAGTTTTTGA | |
| cassette | GTTGTGTATAACCCCTCATTCTGATCCCAGCTTATACGGTCCAGGATCACCGATCATTCACAGTTAAT | |
| | GATCCTTTCCAGGTTGTTGATCTTAAAAGCCGGATCCTTGTTATCCACAGGGCAGTGCGATCGTAATA | |
| | AGAGATCACAATAGAACAGATCTCTAAATAAATAGATCTTCTTTTTAATACTTTAGTTACAACATACT | |
| | ttttggtcatgagattatcaaaaaggatcttcacctagat | |
| oriC_ | aagtggtcctgcaactttatccgcctccatccagtctattaattgttgccgggaagctagagtaagta | 15 |
| 1760 | gttcgccagttaatagtttgcgcaacgttgttgccattgctacaggcatcgtACCGACAGATGTATGT | |
| cassette | AAGGCCAACGTGCTCAAATCTTCATACAGAAAGATTTGAAGTAATATTTTAACCGCTAGATGAAGAGC | |
| | AAGCGCATGGAGCGACAAAATGAATAAAGAACAATCTGCTGATGATCCCTCCGTGGATCTGATTCGTG | |
| | TAAAAAATATGCTTAATAGCACCATTTCTATGAGTTACCCTGATGTTGTAATTGCATGTATAGAACAT | |
| | AAGGTGTCTCTGGAAGCATTCAGAGCAATTGAGGCAGCGTTGGTGAAGCACGATAATAATATGAAGGA | |
| | TTATTCCCTGGTGGTTGACTGATCACCATAACTGCTAATCATTCAAACTATTTAGTGTGTGACAGAGC | |
| | CAACACGCAGTCTGTCACTGTCAGGAAAGTGGTAAAACTGCAACTCAATTACTGCAATGCCCTCGTAA | |
| | TTAAGTGAATTTACAATATCGTCCTGTTCGGAGGGAAGAACGCGGGATGTTCATTCTTCATCACTTTT | |
| | AATTGATGTATATGCTGTCTTTTCTGACGTTAGTCTCCGACGGCAGGGTTCAATGACCCAGGGTGAGA | |
| | AATTCCCGGACCCTTTTTGCTCAAGAGCGATGTTAATTTGTTCAATCATTTGGTTAGGAAAGCGGATG | |
| | TTGCGGGTTGTTGTTCTGCGGGTTCTGTTCTTCGTTGACATGAGGTTGCCCCGTATTCAGTGTCGCTG | |
| | ATTTGTATTGTCTGAAGTTGTTTTTACGTTAAGTTGATGCAGATCAATTAATACGATACCTGCGTCAT | |
| | AATTGATTATTTGACGTGGTTTGATGGCCTCGACGCACGTTGTGATATGTAGATGATAATCATTATCA | |
| | CTTTACGGGTCCTTTCCGGTGATCCGACAGGTTACGGGGCGGCGACCTAGTATGTTGTAACTAAAGAT | |
| | CTACTGTGGATAACTCTGTCAGGAAGCTTGGATCAACCGGTAGTTATCCAAAGAACAACTGTTGTTCA | |
| | GTTTTTGAGTTGTGTATAACCCCTCATTCTGATCCCAGCTTATACGGTCCAGGATCACCGATCATTCA | |
| | CAGTTAATGATCCTTTCCAGGTTGTTGATCTTAAAAGCCGGATCCTTGTTATCCACAGGGCAGTGCGA | |
| | TCCTAATAAGAGATCACAATAGAACAGATCTCTAAATAAATAGATCTTCTTTTTAATACTTTAGTTAC | |
| | AACATACTGGGCGGCGACCTCGCGGGTTTTCGCTATTTATGAAAATTTTCCGGTTTAAGGCGTTTCCG | |
| | TTCTTCTTCGTCATAACTTAATGTTTTTATTTAAAATACCGTCTGAAAAGAAAGGAAACGACAGGTGC | |
| | TGAAAGCGAGGCTTTTTGGCCTCTGTCGTTTCCTTTCTCTGTTTTTGTCCGTGGAATGAACAATGGAA | |
| | GTCAACAAAAAGCAGCTGGCTGACATTTTCGGTGCGAGTATCCGTACCATTCAGAACTGGCAGGAACA | |
| | GGGAATGCCCGTTCTGCGAGGCGGTGGCAAGGGTAATGAGGTGCTTTATGACTCTGCCGCCGTCATAA | |
| | AATGGTATGCCGAAAGGGATGCTGAAATTGAGAACGAAAAGCTGCGCCGGGAGGTTGAAGAACTGCGG | |
| | CAGGCCAGCGAGGCAGATCTCCAGCCAGGAACTATTGAGTACGAACGCCATCGACTTACGCGTGCGCA | |
| | GGCCGACGCACAGGAACTGAAGAATGCCAGAGACTCCGCTGAAGTGGTGGAAACCGCATTCTGTACTT | |
| | TCGTGCTGTCGCGGATCGCAGGTGAAATTGCCAGTATTCTGGACGGGCTCCCCCTGTCGGTGCAGCGG | |
| | CGTTTTCCGGAACTGGAAAACCGACATGTTGATTTCCTGAAACGGGATATCATCAAAGCCATGAACAA | |
| | AGCAGCCGCGCTGGATGAACTGATACCGGGGTTGCTGAGTGAATATATCGAACAGTCAGGTTAACAGG | |
| | CTGCGGCATTTTGTCCGCGCCGGGCTTCGCTCACTGTTCAGGCCGGAGCCACAGACCGCCGTTGAATG | |
| | GGCGGATGCTAATTACTATGTCCCGAAAGAATCCGCATACGAGGAAGGGCGCTGGGAAACACTGCCCT | |
| | TTCAcgctcgtcatcaaaatcactcgcatcaaccaaaccgttattcattcgtgattgcgcctgagcga | |
| | gacgaaatacgcgatcgctgttaaaaggacaattacaaacaggaatcgaatgcaac | |

Homologous recombination and RCR amplification were performed in the same manner as Example 1 other than adding 400 pM of pUC4K and 400 pM of the oriC_pUCori2 cassette, or 180 pM of pUC4K and 180 pM of the oriC_1760 cassette, to the homologous-recombination reaction solution. The obtained amplification product was subjected to electrophoresis, and the bands were stained.

The staining results are shown in FIG. 9. In the figure, the “insertion” lane is a lane where the homologous-recombination reaction solution of pUC4K and the oriC_pUCori2 cassette is subjected to electrophoresis, and the “replacement” lane is a lane where the homologous-recombination reaction solution of pUC4K and the oriC_1760 cassette is subjected to electrophoresis.

When homologous recombination is performed by using pUC4K and the oriC_pUCori2 cassette, circular DNA in which 300 bp from the oriC_pUCori2 cassette are inserted between the region Ha and the region Hb of pUC4K—that is, circular DNA that is 300 bp longer than the pre-reaction pUC4K—is obtained. When homologous recombination is performed by using pUC4K and the oriC_1760 cassette, circular DNA in which a 1,760 bp region that is sandwiched by the region Ha and the region Hb in pUC4K is substituted with 2,060 bp region from the oriC_1760 cassette—that is, circular DNA that is 300 bp longer than the pre-reaction pUC4K—is obtained. In fact, as shown in FIG. 9, amplification-product bands were confirmed in the same position, as predicted from homologous recombination, in both the “insertion” and “replacement” lanes. Upon subjecting the amplification product to restriction-enzyme mapping that uses the restriction enzyme PstI or EcoRV, the amplification product was confirmed to be the circular DNA that is being sought. It was confirmed from the results that even a long linear-DNA fragment of 2 kbp can be recombined into circular DNA by homologous recombination as long as a RecA-family-recombinase protein and an exonuclease are present.

### Example 9

In homologous recombination in which a linear double-stranded DNA fragment is inserted into circular double-stranded DNA by using a RecA-family-recombinase protein and a 3′ to 5′ exonuclease, the efficiencies of insertion into open circular DNA (oc) that has nicks and into supercoiled DNA (sc) that does not have nicks were examined.

The plasmid pCoco20k (total length of 19.7 kb) or the plasmid pCoco30k (total length of 30.2 kb) was used as the circular double-stranded DNA. pCoco20k was constructed by Escherichia coli cloning by subjecting 10.2 kb of an Escherichia coli genome region to ligation and circularization with a 9.4 kb fragment that includes the replication origin of the plasmid pETcoco-2 (manufactured by Novagen) and a drug resistance gene. pCoco30k was constructed by Escherichia coli cloning by subjecting 20.7 kb of an Escherichia coli genome region to ligation and circularization by the same method as the method used for pCoco20k. 60 bp regions that are adjacent to each other were set as the region Ha and the region Hb in each plasmid.

Linear double-stranded DNA fragments (oriC2.0-BAC20k cassette and oriC2.0-BAC30k cassette) that have base sequences (SEQ ID NO: 16 and SEQ ID NO: 17) in which the oriC sequence is ligated downstream of a base sequence that is identical to the region Ha and in which a base sequence that is identical to the region Hb is ligated downstream of the oriC sequence were used as the linear double-stranded DNA fragments that are respectively inserted into pCoco20k and pCoco30k. Both cassettes were made to have a size of 546 bp. Table 8 indicates the base sequences of the oriC2.0-BAC20k cassette and the oriC2.0-BAC30k cassette. In the base sequences of SEQ ID NO: 16 and SEQ ID NO: 17 in table 8, the upstream lowercase region (5′ end side) is the base sequence that is identical to the region Ha, and the downstream lowercase region (3′ end side) is the base sequence that is identical to the region Hb. Only the underlined regions differ between the base sequences.

TABLE 8

| | | SEQ |
|---|---|---|
| | Base sequence | ID NO: |
| ori02.0_ | tgggaccacggtcccacttgtattgtcgatcagactatcagcgtgagactacgattccatT | 16 |
| BAC20k | AAAAAAACAGCCTCCGAAAGGAGGCTGTAGGCAAATATTCTAGTATGTTGTACCTAAAGGA | |
| cassette | AATGAGCTGACAGATCGTGCGATCTACTGTGGATAACTCTGTCAGGAAGCTTGGATCAACC | |
| | GGTAGTTATCCAAAGAACAACTGTTGTTCAGTTTTTGAGTTGTGTATAACCCCTCATTCTG | |
| | ATCCCAGCTTATACGGTCCAGGATCACCGATCATTCACAGTTAATGATCCTTTCCAGGTTG | |
| | TTGATCTTAAAAGCCGGATCCTTGTTATCCACAGGGCAGTGCGATCCTAATAAGAGATCAC | |
| | AATAGAACAGATCTCTAAATAAATAGATCTTCTTTTTAATACCCAGGATCCCTTTAGGTAC | |
| | AACATACTGTTTGGGATTCACTGCATTATAGTGTGAAAATGACCCTCTGTCAACAGCTCat | |
| | gtcgtcgtaacctgtagaacggagttcttttccgcgggctgggatttaaaagcggcag | |
| oriC2.0_ | tgggaccacggtcccacttgtattgtcgatcagactatcagcgtgagactacgattccatT | 17 |
| BAC30k | AAAAAAACAGCCTCCGAAAGGAGGCTGTAGGCAAATATTCTAGTATGTTGTACCTAAAGGA | |
| cassette | AATGAGCTGACAGATCGTGCGATCTACTGTGGATAACTCTGTCAGGAAGCTTGGATCAACC | |
| | GGTAGTTATCCAAAGAACAACTGTTGTTCAGTTTTTGAGTTGTGTATAACCCCTCATTCTG | |
| | ATCCCAGCTTATACGGTCCAGGATCACCGATCATTCACAGTTAATGATCCTTTCCAGGTTG | |
| | TTGATCTTAAAAGCCGGATCCTTGTTATCCACAGGGCAGTGCGATCCTAATAAGAGATCAC | |
| | AATAGAACAGATCTCTAAATAAATAGATCTTCTTTTTAATACCCAGGATCCCTTTAGGTAC | |
| | AACATACTGTTTGGGATTCACTGCATTATAGTGTGAAAATGACCCTCTGTCAACAGCTCat | |
| | gtcgtcgtaacctgtagaacggagtaacctcggtgtgcagattgtttccctccgtaaa | |

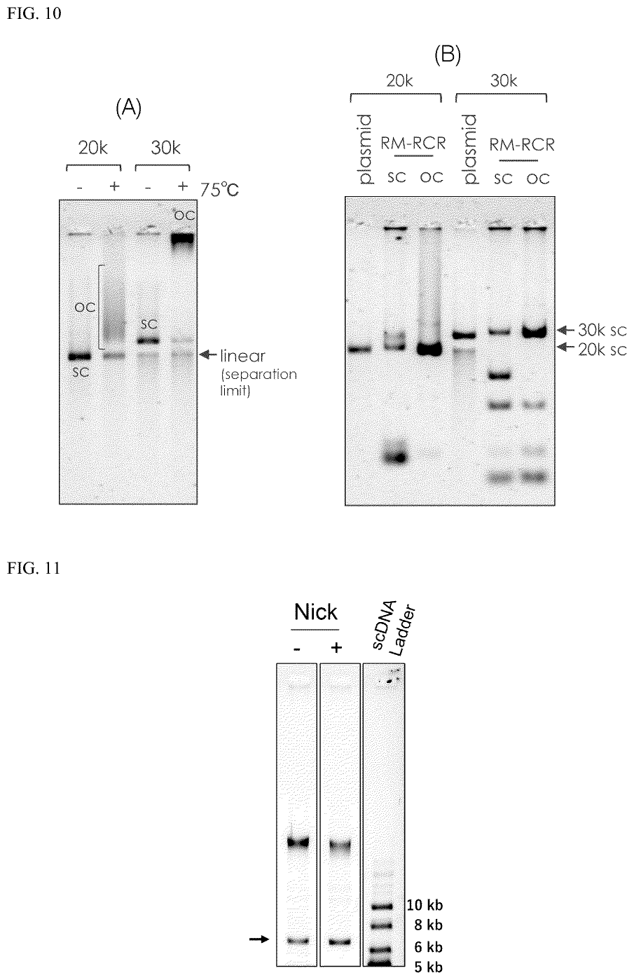

Circular DNA (oc) was obtained by processing supercoiled DNA (sc) of pCoco20k and pCoco30k for 1 hour at 75° C. to form nicks in each plasmid. (A) in FIG. 10 shows the staining image of the bands that were separated by subjecting pCoco20k and pCoco30k, both before and after the 75° C. processing, to agarose electrophoresis. In the figure, “20k” indicates the lane where pCoco20k is subjected to electrophoresis, and “30k” indicates the lane where pCoco30k is subjected to electrophoresis. That the plasmids after the 75° C. processing include open circular DNA (oc) in which nicks are formed was confirmed as a result.

Homologous recombination and RCR amplification were performed in the same manner as Example 1 other than adding 80 pM of pCoco20k and 80 pM of the oriC2.0-BAC20k cassette, or 80 pM of pCoco30k and 80 pM of the oriC2.0-BAC30k cassette, to the homologous-recombination reaction solution, changing the incubation temperature in homologous recombination from 37° C. to 42° C., and incubating the RCR amplification for 6 hours at 33° C. instead of 16 hours at 30° C. The obtained amplification product was subjected to electrophoresis, and the bands were stained. The pCoco20k and the pCoco30k were examined as supercoiled DNA (sc) and as open circular DNA (oc) with nicks. As the control for size comparison, the circular double-stranded DNA prior to having the linear double-stranded DNA fragment inserted therein was subjected to electrophoresis and stained in the same manner.

The staining results are shown in (B) in FIG. 10. As a result, a DNA band in a position that is slightly larger than the size from before homologous recombination was detected for both pCoco20k and pCoco30k when homologous recombination was performed, confirming that a product into which the linear DNA is inserted is obtained. The open circular DNA (oc) with nicks had a higher quantity and purity of the insertion product than the pre-reaction supercoiled DNA (sc). Thus, it was confirmed that using the open circular DNA (oc) causes more efficient homologous recombination.

Moreover, upon carrying out the same reaction by using a fragment that has a drug resistance gene as the linear double-stranded DNA fragment, it was confirmed that a plasmid that is added with new drug resistance is obtained.

### Example 10

In homologous recombination in which a linear double-stranded DNA fragment is inserted into circular double-stranded DNA, the region from the region Ha to the region Hb in the circular double-stranded DNA was substituted with the linear double-stranded DNA fragment by making the ends of the linear double-stranded DNA fragment into single strands by using the USER (registered trademark) enzyme, without using an exonuclease.

pSV-β-galactosidase (Promega, size of 6.8 kb) was used as the circular double-stranded DNA, and a 75 bp region and an 80 bp region that are adjacent to each other were respectively set as the region Ha and the region Hb.

An oriC-SV cassette (size of 487 bp) that has a base sequence (SEQ ID NO: 18) in which the oriC sequence is ligated downstream of a base sequence that is identical to the region Ha and in which a base sequence that is identical to the region Hb is ligated downstream of the oriC sequence was used as the linear double-stranded DNA fragment. The base sequence of the oriC-SV cassette is shown in table 9. In the base sequence of SEQ ID NO: 18 in table 9, the upstream lowercase region (5′ end side) is the base sequence that is identical to the region Ha, and the downstream lowercase region (3′ end side) is the base sequence that is identical to the region Hb.

TABLE 9

| | | SEQ ID |
|---|---|---|
| | Base sequence | NO: |
| oriC-SV | ttttgataatctcatgaccaaaatcccttaacgtgagttttcgttccactgagcgtcagacc | 18 |
| cassette | ccgtagaaaagatGCACACCGAGGTTACTCCGTTCTACGGGATCCTGGGTATTAAAAAGAAG | |
| | ATCTATTTATTTAGAGATCTGTTCTATTGTGATCTCTTATTAGGATCGCctaGgCCTGTGGA | |
| | TAACctaGgTCCGGCTTTTAAGATCAACAACGTGGAAAGGATCATTAACTGTGAATGATCGG | |
| | TGATCCTGGACCGTATAAGCTGGGATCAGAATGAGGGGTTATACACAACTCAAAAACTGAAC | |
| | AACAGTTGTTCTTTGGATAACTACCGGTTGATCCAAGCTTCCTGACAGAGTTATCCACAGTA | |
| | GATCGCACGATCTGTCTTCGTCCATTTGACAGGCCatcaaaggatcttcttgagatcctttt | |
| | tttctgcgcgtaatctgctgcttgcaaacaaaaaaaccaccgctaccagcggt | |
| FW primer | ttttgataatcucatgaccaaaaucccttaacgugagttttcgtuccactgagcgucagacc | 19 |
| | ccguagaaaagauGCACACCGAGGTTACTCCGTTCTAC | |
| RV primer | accgcuggtagcgguggtttttttgttugcaagcagcagatuacgcgcagaaaaaaaggauc | 20 |
| | tcaagaagatcctttgauGGCCTGTCAAATGGACGAAG | |

The oriC-SV cassette was subjected to PCR amplification by using a primer pair that includes dUTP (a pair of a forward primer of the base sequence represented by SEQ ID NO: 19 and a reverse primer of the base sequence represented by SEQ ID NO: 20) to prepare an oriC-SV-USER cassette that includes dUTP at the homologous ends. The underlined portions of the base sequences of the forward primer and the reverse primer in table 9 indicate the dUTP. The lowercase region of the forward primer has the same base sequence as the region Ha other than a portion of the bases being substituted with dUTP. The lowercase region of the reverse primer is a base sequence that is complementary to the base sequence of the region Hb other than a portion of the bases being substituted with dUTP.

Next, 10 ng of the oriC-SV-USER cassette was warmed for 30 minutes at 37° C. in 2 mM of ATP, 20 mU/μL of the Thermolabile USER II enzyme (manufactured by New England BioLabs; an enzyme that includes uracil-DNA glycosylase and endonuclease VIII and creates a one-base gap in the dUTP portion), and the CutSmart buffer (50 mM of potassium acetate, 20 mM of tris acetate, 10 mM of magnesium acetate, and 100 μg/mL of BSA; pH of 7.9 (25° C.)) (manufactured by New England BioLabs) (total amount of 10 μL) that contains 2 μM of RecA (same as the RecA in Example 1) to create a one-base gap in the dUTP portion, make a single-strand overhang in the homologous-end portion by thermal dissociation of the DNA region between the gaps, and form a RecA filament in the single-strand overhang. 0.5 μL of the reaction solution was added to the CutSmart buffer (total amount of 5 μL), the buffer containing 5 ng of pSV-β-galactosidase. The mixture was warmed for 10 minutes at 37° C., and homologous recombination was performed. As the pSV-β-galactosidase, unprocessed supercoiled DNA (−) and open circular DNA (+) that has nicks as in Example 9 were used.

RCR amplification was performed for 16 hours at 30° C. as in Example 1 to confirm the insertion of the oriC-SV-USER cassette. After the end of the RCR amplification, a portion (0.4 μL) of the RCR-amplification reaction solution was diluted to one-tenth in an RCR buffer. The diluted solution was then incubated for 30 minutes at 30° C. Afterward, 1 μL of the diluted solution was subjected as-is to agarose electrophoresis, and the separated bands were stained by using SYBR Green. As a size marker, a supercoiled DNA (scDNA) ladder (manufactured by New England BioLabs) was subjected to electrophoresis and stained at the same time.

The results are shown in FIG. 11. RCR amplification of scDNA of the predicted size was detected independently of the presence or absence of nicks in the circular double-stranded DNA (see the arrow in FIG. 11). Making the ends of the linear double-stranded DNA fragment into single strands by using the USER enzyme, without using an exonuclease, also enabled the region from the region Ha to the region Hb in the circular double-stranded DNA to be substituted with the linear double-stranded DNA fragment.

1. A method for producing circular DNA in which a region that is sandwiched by a region Ha and a region Hb in circular double-stranded DNA is substituted with the entirety or a portion of a linear-DNA fragment, wherein
the region Hb is located downstream of the region Ha in the circular double-stranded DNA;
the linear-DNA fragment is single-stranded or double-stranded linear DNA that has a homologous region that corresponds to the region Ha and a homologous region that corresponds to the region Hb, the latter homologous region being positioned downstream of the former homologous region; and the method comprising:
preparing a reaction solution that contains the circular double-stranded DNA, the linear-DNA fragment, and a protein that has RecA-family-recombinase activity, and
performing homologous recombination reaction by incubating the reaction solution for a predetermined period of time,
thereby producing circular DNA in which the region that is from the region Ha to the region Hb in the circular double-stranded DNA is substituted with the region that is from the homologous region that corresponds to the region Ha to the homologous region that corresponds to the region Hb in the linear-DNA fragment.

2. The method for producing circular DNA according to claim 1, wherein the linear-DNA fragment is double-stranded linear DNA, and at least a portion of the linear-DNA fragment is made into a single strand at the same time as or prior to the homologous recombination.

3. The method for producing circular DNA according to claim 1, wherein the base-pair length of the region Ha and the base-pair length of the region Hb are 10 to 500 bp, respectively.

4. The method for producing circular DNA according to claim 1, wherein the reaction solution is incubated in a temperature range of 20 to 48° C. in the homologous recombination.

5. The method for producing circular DNA according to claim 1, wherein the circular double-stranded DNA prior to the homologous recombination has a nick.

6. The method for producing circular DNA according to claim 1, wherein the linear-DNA fragment is double-stranded linear DNA, and the reaction solution further contains an exonuclease.

7. The method for producing circular DNA according to claim 6, wherein the exonuclease is a 3′ to 5′ exonuclease or a 5′ to 3′ exonuclease.

8. The method for producing circular DNA according to claim 6, wherein the reaction solution contains an enzyme for regenerating a nucleoside triphosphate or deoxynucleotide triphosphate and a substrate for the enzyme.

9. The method for producing circular DNA according to claim 8, wherein:
the enzyme is creatine kinase and the substrate is creatine phosphate,
the enzyme is pyruvate kinase and the substrate is phosphoenolpyruvate,
the enzyme is acetate kinase and the substrate is acetyl phosphate,
the enzyme is a polyphosphate kinase and the substrate is a polyphosphate, or
the enzyme is a nucleoside-diphosphate kinase and the substrate is a nucleoside triphosphate.

10. The method for producing circular DNA according to claim 1, wherein the circular DNA that is obtained by the homologous recombination is amplified.

11. The method for producing circular DNA according to claim 1, wherein
the circular DNA that is obtained by the homologous recombination contains a replication origin that can bind to an enzyme that has DnaA activity, and the method comprising:
forming a reaction mixture that contains the circular DNA that is obtained by the homologous recombination, a first enzyme group that catalyzes replication of circular DNA, a second enzyme group that catalyzes Okazaki fragment joining reaction and synthesizes two sister circular DNAs constituting a catenane, and a third enzyme group that catalyzes the separation of the two sister circular DNAs, and dNTP; and
incubating the reaction mixture formed under an isothermal conditions;
thereby repairing a gap and nick in the circular DNA that is obtained by the homologous recombination and amplifying the circular DNA.

12. The method for producing circular DNA according to claim 1, wherein
the linear-DNA fragment has a target gene between the homologous region that corresponds to the region Ha and the homologous region that corresponds to the region Hb, and
circular DNA is produced that has the target gene inserted in the region that is sandwiched by the region Ha and the region Hb in the circular double-stranded DNA.

13. The method for producing circular DNA according to claim 12, wherein the target gene is a drug resistance gene.

14. The method for producing circular DNA according to claim 12, wherein the circular double-stranded DNA is a plasmid.
